(19) United States
(12) Patent Application Publication
(10) Pub. No.: US 2011/0189095 A1
(43) Pub. Date: Aug. 4, 2011

(54) CRKL TARGETING PEPTIDES

(75) Inventors: Wadih Arap, Houston, TX (US); Paul J. Miniz, London (GB); Renata Pasqualini, Houston, TX (US)

(73) Assignee: The Board of Regents, of the University of Texas System, Austin, TX (US)

(21) Appl. No.: 13/000,231

(22) PCT Filed: Jun. 19, 2009
(86) PCT No.: PCT/US09/48024

§ 371 Date: Mar. 25, 2011

Related U.S. Application Data
(60) Provisional application No. 61/074,423, filed on Jun. 20, 2008.

(51) Int. Cl. A61K 38/02; C07K 2/00; C07K 7/08; C07K 14/47; C07K 7/06; C07K 19/00; C07K 16/00; C07K 14/555; C07K 14/54; C12N 9/96; C07K 14/545; C07K 14/55; C07K 14/57; C07K 14/565; C07K 14/56; C07K 14/525; C07K 14/535; C12N 7/00; C12N 5/071; C12N 1/20; C12N 1/16; C07K 1/04; C12N 5/07; C12Q 1/02; A61P 35/00; A61P 35/02; A61K 49/00

(52) U.S. Cl. 424/9.1; 530/300; 530/326; 530/324; 530/328; 530/317; 530/350; 530/409; 530/391.7; 530/351; 530/322; 435/188; 435/235.1; 435/325; 435/252.1; 435/255.1; 530/345; 435/375; 514/1.1; 435/29; 514/19.3; 514/19.4; 514/19.5; 514/19.6

(57) ABSTRACT

Provided are methods and compositions for selectively targeting CRKL through the use of targeting peptides. Selective targeting of secreted CRKL through the use of a targeting peptide may be used, for example, in the treatment of cancer to deliver a chemotherapeutic compound, fusion protein, or fusion construct to a cancer cell or tissue.

This application claims priority to U.S. Application No. 61/074,423 filed on Jun. 20, 2008, the entire disclosure of which is specifically incorporated herein by reference in its entirety without disclaimer.

This invention was made with U.S. government support under grants PC050442 from the National Institutes of Health and Department of Defense. The government has certain rights in the invention.

### BACKGROUND OF THE INVENTION

1. Field of the Invention

The present invention concerns the fields of molecular medicine and targeted delivery of therapeutic and detecting agents. More specifically, the present invention relates to the identification of novel peptide sequences that selectively target cancers for the treatment and detection of cancer.

2. Description of Related Art

Therapeutic treatment of many human disease states is limited by the systemic toxicity of the therapeutic agents used. Cancer therapeutic agents in particular exhibit a very low therapeutic index, with rapidly growing normal tissues such as skin and bone marrow typically affected at concentrations of agent that are not much higher than the concentrations used to kill tumor cells. Treatment and diagnosis of cancer would be greatly facilitated by the development of compositions and methods for targeted delivery to cancer cells, more specifically, by using antibodies or tumor-homing peptides that bind to targets found on the surfaces of cancer cells, but not found on normal tissues. Such targets, which must have minimal homology to other cell surface molecules, are difficult to find.

Recently, an in vivo selection system was developed using phage display libraries to identify organ or tissue targeting peptides in a mouse model system. Such libraries can be generated by inserting random oligonucleotides into cDNAs encoding a phage surface protein, generating collections of phage particles displaying unique peptides in as many as 10e9 permutations (Pasqualini and Ruoslahti, 1996, Arap et al., 1998; Pasqualini et al., 2001). Intravenous administration of phage display libraries to tumor bearing mice was followed by the recovery of phage from tumor xenograft and the tumor targeting peptides capable of selective homing to the tumor were characterized. Phage were recovered that were capable of selective homing to the vascular beds of different mouse organs or tissues, based on the specific targeting peptide sequences expressed on the outer surface of the phage (Pasqualini and Ruoslahti, 1996). Each of those tumor-homing peptides bound to receptors that were selectively expressed or upregulated in the surface of tumor cells.

Attachment of therapeutic agents to targeting peptides resulted in the selective delivery of the agent to a desired organ or tissue in the mouse model system. Targeted delivery of chemotherapeutic agents and proapoptotic peptides to receptors located in tumor angiogenic vasculature resulted in a marked increase in therapeutic efficacy and a decrease in systemic toxicity in tumor-bearing mouse models (Arap et al., 1998a, 1998b; Ellerby et al., 1999).

CRKL (chicken tumor virus number 10 regulator of kinase-like protein), an adapter protein, is a homolog of oncogene v-crk. It contains one SH2 domain and two tandem SH3 domains. Intracellular CRKL is implicated in both MAP kinase and integrin-mediated pathways (Li et al., 2003; Uemura et al., 1999). In addition, CRKL has an oncogenic potential.

Presently, cancer patients are routinely treated with systematic chemotherapy and radiotherapy. However, such treatments are often plagued by well-known side-effects and limited efficacy. There is clearly a need for new compositions and methods for targeted delivery of therapeutic and diagnostic agents.

### SUMMARY OF THE INVENTION

The present invention overcomes deficiencies in the prior art by providing methods and compositions for selectively targeting secreted CRKL through the use of targeting peptides. Selective targeting of secreted CRKL through the use of a targeting peptide may be used, for example, in the treatment of cancer to deliver a chemotherapeutic compound, fusion protein, or fusion construct to a cancer cell or tissue.

To gain insight in mechanisms of signal transduction across cell membranes in cancer, the inventors set out to uncover functional protein interactions in a tumor xenograft model. The inventors reasoned that a combinatorial approach (Hajitou et al., 2006; Arap et al., 2002; Arap et al., 1998; Arap et al., 2004; Pasqualini and Ruoslahti, 1996) such as serial selection from phage display random peptide libraries in vivo might provide clues by emulating unbiased ligand-receptor binding within the context of the tumor microenvironment. As shown in the below examples, a specific interaction between the intracellular signaling protein CRKL and a regulatory (rather than ligand-binding) β₁ integrin extracellular domain was observed. Surprisingly, the inventors found that CRKL targets the plexin-semaphorin-integrin (PSI) domain of β₁ integrin located outside of the cell, triggers MAP kinases, and promotes cell growth and survival. Without wishing to be bound by any theory, these results support the idea that an unrecognized integrin-mediated outside-in function exists for intracellular mediators, such as SH3-containing proteins, in activating the MAP kinase pathway.

An aspect of the present invention related to an isolated tumor targeting peptide comprising a CRKL binding motif, said motif defined as: being from 6 to 20 amino acids in length, having a degree of similarity to a corresponding best fit sequence alignment to β1 integrin (SEQ ID NO:47) of at least 25%; and wherein the targeting peptide is 100 amino acids or less in length and binds under physiological conditions to cells expressing CRKL. The CRKL binding motif may have a degree of similarity to a best fit sequence alignment to β1 integrin (SEQ ID NO:47) of at least 40%, at least 50%, or at least 60%. In certain embodiments, the peptide has a sequence that is not identical to a best fit sequence alignment to β1 integrin (SEQ ID NO:47). In certain embodiments, the CRKL binding motif may have a best fit sequence alignment to a β1 integrin (SEQ ID NO:47) PSI domain region. The CRKL binding motif may have a best fit sequence alignment to a β1 integrin (SEQ ID NO:47) PSI domain region selected from the group consisting of amino acids 6 to 10, 10 to 29; 15 to 34; 18 to 37; 36 to 55; 39 to 58; 45 to 64; 94 to 113; 196 to 215; 198 to 213; 203 to 222; 244 to 263; 330 to 349; 377 to 396; 379 to 398; 380 to 399; 398 to 417; 400 to 419; 413 to 432; 447 to 466; 460 to 479; 460 to 479; 464 to 483; 469 to 488; 474 to 493; 475 to 494; 512 to 533; 519 to 538; 551 to 570; 574 to 593; 577 to 596; 579 to 598; 590 to 609; 596 to 615; 613 to 632; 615 to 634; 616 to 635; 644 to 663; 648 to 667; 663 to 682; 674 to 693; 682 to 701; 721 to 740; 727 to 746; and 779 to 798. In certain embodiments, the CRKL binding motif has a sequence selected from the group consisting of SEQ ID NO:1 through SEQ ID NO:46.

In certain embodiments, the isolated peptide may be further defined as a cyclic peptide that are capable of being prepared in a cyclic form, such as peptide having a cysteine residue (“C”) at both termini, which may, where desired, be provided in cyclic form, such as through the formation of a di-cysteine (i.e., cystine). Such cyclic peptides may be of particular importance in that disulfide bonds in peptides makes them remarkably stable to chemical, thermal or enzymatic degradation. Such cyclic peptides may be of particular importance in therapeutic and diagnostic applications, where poor availability, susceptibility to proteolysis and short in vivo half-lives are concerned.

Said peptide may be attached to a molecule; for example, the molecule may be a protein and the peptide may be conjugated or fused to the protein to form a protein conjugate, wherein the protein conjugate is not a naturally occurring protein. The peptide may be positioned at a terminus of the protein. Said molecule may be a pro-apoptosis agent, an anti-angiogenic agent, a cytokine, a cytotoxic agent, a drug, a chemotherapeutic agent, a hormone, a growth factor, an antibiotic, an antibody or fragment or single chain thereof, a survival factor, an anti-apoptotic agent, a hormone antagonist, an antigen, a peptide, a protein, a diagnostic agent, a radioisotope, or an imaging agent. Said molecule may be a pro-apoptosis agent selected from the group consisting of gramicidin; magainin; mellitin; defensin; cecropin; (KLAKLAK)₂ (SEQ ID NO:48); (KLAKKLA)₂ (SEQ ID NO:49); (KAAKKAA)₂ (SEQ ID NO:50); (KLGKKLG)₃ (SEQ ID NO:51); Bcl-2; Bad; Bak; Bax; and Bik. In certain embodiments, said pro-apoptosis agent is (KLAKLAK)₂ (SEQ ID NO:48). SEQ ID NO:48 may consist of D amino acids.

In other embodiments, said molecule may be an anti-angiogenic agent selected from the group consisting of thrombospondin, an angiostatin, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4, minocycline, endostatin XVIII, endostatin XV, the C-terminal hemopexin domain of matrix metalloproteinase-2, the kringle 5 domain of human plasminogen, a fusion protein of endostatin and angiostatin, a fusion protein of endostatin and the kringle 5 domain of human plasminogen, the monokine-induced by interferon-gamma (Mig), a fusion protein of Mig and IP10, soluble FLT-1 (fins-like tyrosine kinase 1 receptor), or kinase insert domain receptor (KDR). Said molecule may be a cytokine selected from the group consisting of interleukin 1 (IL-1), IL-2, IL-5, IL-10, IL-11, IL-12, IL-18, interferon-γ (IF-γ), IF-α, IF-β, a tumor necrosis factor, or GM-C SF (granulocyte macrophage colony stimulating factor).

Said peptide may be attached to a macromolecular complex, such as a virus, a bacteriophage, a bacterium, a liposome, a microparticle, a magnetic bead, a yeast cell, or a mammalian cell. In certain embodiments, said peptide is attached to a virus, such as a lentivirus, papovavirus, adenovirus, retrovirus, AAV, vaccinia virus or herpes virus. Said peptide may be attached to a solid support, such as a microtiter dish or microchip.

Another aspect of the present invention relates to a method of preparing a construct comprising obtaining a peptide in accordance with the present invention and attaching the peptide to a molecule to prepare the construct.

Yet another aspect of the invention relates to a method of targeting the delivery of a peptide, molecule or protein to cells that express CRKL, the method comprising the steps of: obtaining a peptide according to the present invention, or prepared by the above method, and administering the peptide to a cell population, wherein the population includes cells that express CRKL, to thereby deliver the molecule or protein to said cells. The cells that express CRKL may be in a subject, and the peptide or protein fusion construct may be formulated in a pharmaceutically acceptable composition and the composition may be administered to the subject. The subject may be a human subject. In certain embodiments, the method is further defined as a detection method and the method further comprises detecting the peptide, molecule or protein that has been delivered to the cells. The subject may have a disease or disorder and the method may be further defined as a therapeutic method. The subject may have a cancer, such as a cancer of the prostate, breast, sarcoma, gum, tongue, lung, skin, liver, kidney, eye, brain, leukemia, mesothelioma, neuroblastoma, head, neck, pancreatic, renal, bone, testicular, ovarian, mesothelioma, cervical, gastrointestinal, lymphoma, brain, colon and bladder.

Embodiments discussed in the context of a methods and/or composition of the invention may be employed with respect to any other method or composition described herein. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings are part of the present specification and are included to further demonstrate certain aspects of the present invention. The invention may be better understood by reference to the drawings in combination with the detailed description of specific embodiments presented herein.

FIG. 1A-C: Peptide targeting and internalization in cancer cells. FIG. 1A, the phage peptide YRCTLNSPFFWEDMTHECHA (SEQ ID NO:1) binds to the cell surface on DU145 cells. A phage clone displaying the sequence RGD-4C (Arap et al., 1998) served as a positive control and fd-tet (insertless) as a negative control. Bars represent mean±standard deviation from triplicate plating. FIG. 1B, immunolocalization of tumor-homing phage on the cell surface of non-permeabilized KS1767 cells. FIG. 1C,D, Synthetic peptide YRCTLNSPFFWEDMTHECHAGG (SEQ ID NO:67)-_{D}(KLAKLAK)₂, enables internalization within DU145 cells as measured by cell viability by using WST-1 reagent and an anti-annexin-V FITC antibody.

FIG. 2A-C: Sequence alignment of tumor-homing peptides and β₁ integrin (SEQ ID NO:13). FIG. 2A, the tumor-homing peptide YRCTLNSPFFWEDMTHECHA (SEQ ID NO:1) matches to the plexin-semaphorin-integrin (PSI) domain (sequence region 26-78 residues) (SEQ ID NO:12). FIG. 2B, sequence alignment of all eight β integrin-subunits (SEQ ID NO:14-21) and the YRCTLNSPFFWEDMTHECHA (SEQ ID NO:1) peptide sequence. FIG. 2C, sequence alignment of all the tumor homing peptides (Table 1) and β₁ integrin (SEQ ID NO:13).

FIG. 3A-D: Receptor binding by peptides. FIG. 3A, the recombinant His-tag CRKL (rCRKL), rCRKL-SH3 (N) domain, and rCRKL-SH3 (C) domain bind to the tumor-homing peptide-YRCTLNSPFFWEDMTHECHA (SEQ ID NO:1). FIG. 3B, the recombinant His-tag rCRKL, rCRKL-SH3 (N) domain, and rCRKL-SH3 (C) domain bind to the synthetic peptide NSTFLQEGMPTSA (SEQ ID NO:23) corresponding to a region in the PSI domain. FIG. 3C, recombinant gst fusion protein of the entire PSI domain (residues 22-82. SEQ ID NO: 71-CLKANAKSCGECIQAGPNCGWCTNSTFLQEGMPTSARCDDLEALKKKGC), gst-PSI linear (residues 48-62, SEQ ID NO: 72-TNSTFLQEGMPTSAR), gst-PSI cyclic (residues 26-74, SEQ ID NO: 72-CTNSTFLQEGMPTSARC) were generated for binding assays. Both the linear and the cyclic PSI-derived regions bind to CRKL. FIG. 3D, the binding activity of the tumor-homing peptide to rCRKL-SH3 (C) domain is inhibited by the tumor-homing (YRCTLNSPFFWEDMTHECHA; SEQ ID NO:1) peptide, by the PSI-derived (NSTFLQEGMPTSA; SEQ ID NO:23) peptide or tumor-homing phage displaying SEQ ID NO:1. Bars represent mean±standard deviation from triplicate wells.

FIG. 4A-D: The interaction between CRKL and binding peptides. FIG. 4A, the binding properties of the rCRKL-SH3 (C) to the tumor-homing peptide. A Pro→Ala in SEQ ID NO:1 is provided as SEQ ID NO:25. Mutational analysis of the CRKL SH3 (C) domain. FIG. 4B, tumor-homing phage (displaying YRCTLNSPFFWEDMTHECHA; SEQ ID NO:1) and the PSI-derived phage (displaying CNSTFLQEGMPTSAC; SEQ ID NO:23) bind to recombinant CRKL. Bars represent mean±standard deviation from triplicate wells. FIG. 4C, scheme of the CRKL SH3 (C) domain (SEQ ID NO:24) and the control deletion mutants generated as His-tag recombinant proteins. Four mutants were generated and tested: Δ1 (deleted residues 236-256), Δ2 (deleted residues 257-277), Δ3 (deleted residues 278-293), and ΔSH3 (C) (deleted residues 236-293). FIG. 4D, the binding region is located between residues 236-277 of the SH3 (C) domain.

FIG. 5: Protein-protein interaction between CRKL and β1 integrin. A concentration-dependent inhibition of CRKL binding to β₁ integrin by recombinant gst-PSI protein (up to 800 ηm). The integrins αvβ3 and αvβ5 served as controls. Standard deviations of the mean from triplicate wells are shown.

FIG. 6A-B: Cell surface localization of CRKL. FIG. 6A, flow cytometry analysis of CRKL on DU145 cells. Immunolabeling was performed by using monoclonal anti-CRKL (**), anti-β₁ integrin (*), and anti-AHSG antibodies (c, control). FIG. 6B, transmission electron microscopy (TEM) of CRKL showing individual CRKL-gold particles on the cell surface (arrow heads). DU145 cells used in studies were fixed without permeabilization. An anti-CRKL polyclonal antibody was used in studies. Scale bars are indicated.

FIG. 7A-C: CRKL secretion. FIG. 7A, Various cancer cell types cultured in serum free media (SFM) secrete the unphosphorylated form of CRKL. FIG. B,C, CRKL antibody neutralizes the extracellular form of CRKL in the medium and affects cell proliferation and migration. Control antibodies used were as follows: anti-IL11 receptor, anti-AHSG, anti-grb2, anti-α₆ integrin, and pre-immune antibodies. Bars represent mean±standard deviation from duplicate wells.

FIG. 8A-D: The effects of siRNA knockdown of CRKL. Cell proliferation (FIG. 8A) adhesion (FIG. 8B) and migration (FIG. 8C) are shown. Standard deviations of the mean from triplicate wells are shown. FIG. 8D, recombinant CRKL rescues CRKL siRNA knockdown cells in cell proliferation assays. DU145 cells were transfected with CRKL siRNA for 48 hours prior to exogenously adding recombinant CRKL to the wells. Cell proliferation was determined with the WST-1 reagent.

FIG. 9A-D: Tumor targeting and mechanistic model. FIG. 9A, in vitro phage binding of tumor-homing or controls (insertless, mutant (YRCTLNSAFFWEDMTHECHA; SEQ ID NO:25), or scrambled (#1: YRFCTSPFHEWHLENTDMCA; SEQ ID NO:26, #2: YRECTDSPHEFHLWNTMCAF; SEQ ID NO:27)) phage on rCRKL. FIG. 9B-D, in vivo homing of targeted or control phage constructs in mice bearing different tumor types. Tumor-homing phage localized to tumors preferentially when compared to controls. Representative data from two independent experiments are shown.

FIG. 10: Targeting inhibition in tumor-bearing mice. Tumor-homing phage was pre-incubated with control gst or recombinant gst-CRKL prior to administering to nude mice bearing size matched human tumors (DU145-derived). Inhibition was observed in the pre-treated tumor-homing phage by recombinant CRKL. Results from two independent experiments are shown.

FIG. 11: Treatment of tumor xenografts by a synthetic tumor-homing proapoptotic peptide. Cohorts of size-matched nude mice bearing human prostate cancer xenografts (DU145-derived) were used. Markedly reduced tumor growth was observed in tumor-bearing mice treated with synthetic tumor-homing proapoptotic peptide YRCTLNSPFFWEDMTHECHAGG (SEQ ID NO:67)-_{D}(KLAKLAK)₂. Equimolar amounts of YRCTLNSPFFWEDMTHECHA (SEQ ID NO:1) or _{D}(KLAKLAK)₂ showed no differences in tumor volume compared to untreated animals (Student's t-test, p<0.001).

### DETAILED DESCRIPTION OF THE INVENTION

The present invention provides both data supporting the importance and function of intracellular signaling protein CRKL as well as peptides which target CRKL. Cell membranes have evolved to interface a tight and compartmentalized control between the intracellular contents and the extracellular milieu (Conner and Schmid, 2003; Cho and Stahelin, 2005). To maintain such homeostasis, transmembrane receptor families mediate bidirectional signaling across the cell surface through a complex spatial and temporal organization of transduction cascades (Martin et al., 2002; Manning et al., 2002). Thus, location of the proteins involved in signal transduction is central to provide specificity of the cellular responses (Cho and Stahelin, 2005; Mochly-Rosen, 1995). For example, on a prototypical mechanotransduction scenario, cell surface receptors such as integrins undergo conformational changes elicited through ligand-binding to enable a cross-talk with signal transduction cascades such as mitogen-activated protein (MAP) kinase pathways; conventional integrin ligands include extracellular matrix (ECM) proteins to their extracellular domains and cytoskeletal proteins to their intracellular domains (Martin et al., 2002; Manning et al., 2002; Hunter, 2000; Pawson and Scott, 1997; Blume-Jensen and Hunter, 2001).

To gain insight in mechanisms of signal transduction across cell membranes in cancer, the inventors set out to uncover functional protein interactions in a tumor xenograft model. The inventors reasoned that a combinatorial approach (Hajitou et al., 2006; Arap et al., 2002; Arap et al., 1998; Arap et al., 2004; Pasqualini and Ruoslahti, 1996) such as serial selection from phage display random peptide libraries in vivo might provide clues by emulating unbiased ligand-receptor binding within the context of the tumor microenvironment. The below data indicates that a specific interaction exists between the intracellular signaling protein CRKL and a regulatory (rather than ligand-binding) β integrin extracellular domain. Surprisingly, the inventors found that CRKL targets the plexin-semaphorin-integrin (PSI) domain of β integrin located outside of the cell, triggers MAP kinases, and promotes cell growth and survival. These results suggest an unrecognized integrin-mediated outside-in function for intracellular mediators, such as SH3-containing proteins, in activating the MAP kinase pathway.

Consistent with the aggregate functional data presented in this work, it is likely that extracellular CRKL plays an as yet unrecognized role in the tumor microenvironment by triggering cell proliferation and migration. Since the intracellular fraction of CRKL itself is also phosphorylated by the addition of exogenous rCRKL, one might speculate that extracellular CRKL (secreted and/or released) can perhaps function as an autocrine or paracrine factor within tumors. These results establish an unusual novel connection between signaling molecules and cell adhesion receptors, in which their relationship at the cell surface can trigger signaling events from the extracellular milieu.

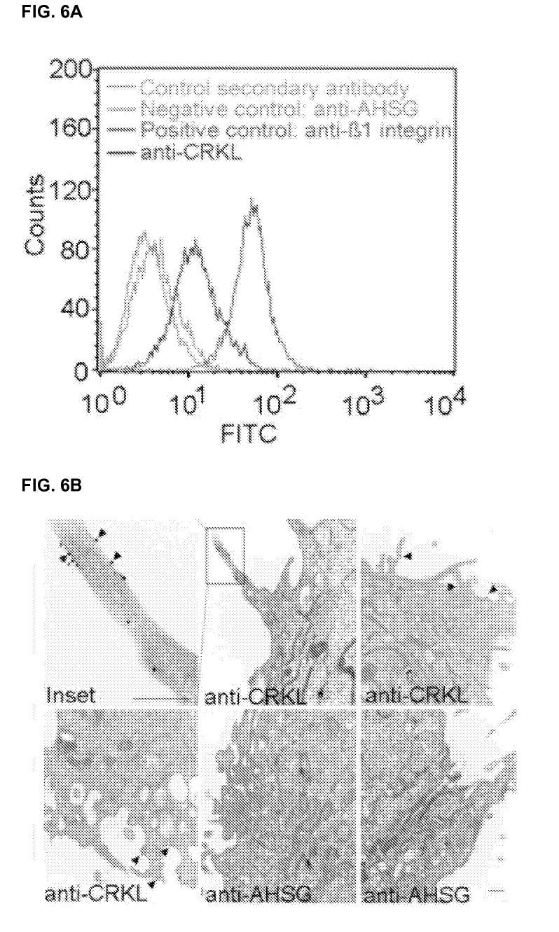

Without wishing to be bound by any theory, based on the “switchblade” structural model for integrin activation (Takagi et al., 2002), the inventors propose an alternative pathway in which extracellular CRKL can activate integrins (from an inactive bent conformation to an active extended conformation) through binding to the PSI domain of the β integrin chain. In the multi-step model presented here (FIG. 6), the below data demonstrates that intracellular unphosphorylated CRKL is either secreted by a non-classic active transport (perhaps via ABC-transporters) and/or released through cell death into the tumor microenvironment (step 1), where its SH3 domains specifically bind to the PSI domain of the β integrin on the tumor cell surface (step 2). Upon binding, the β integrin conformation is changed from bent to extended (active), thus triggering downstream phosphorylation of target proteins in the integrin-mediated pathway (steps 3 and 4) and/or MAP kinase pathway (steps 5-7) and ultimately affecting tumor cell migration and proliferation (step 8). In line with these findings, there have been several recent reports of other intracellular molecules that can also be detected on the cell surface, including various nuclear proteins (Sinclair and O'Brien, 2002; Hovanessian et al., 2000), transcription factors (Monferran et al., 2004), and stress-response chaperones (Arap et al., 2004; Shin et al., 2003; Mintz et al., 2003). Other functional ligand-receptor interactions in addition to the one shown below in the examples may exist between a secreted and/or released signaling molecule acting in the extracellular environment and a cell adhesion receptor and may have general biological significance.

The present invention provides isolated tumor targeting peptides comprising a CRKL binding motif, and said motif defined as being, e.g., from 6 to 20 amino acids in length, having a degree of similarity to a corresponding best fit sequence alignment to β integrin (SEQ ID NO:47) of, e.g., at least 25%; and wherein the targeting peptide may be 100 amino acids or less in length and binds under physiological conditions to cells expressing CRKL. The CRKL binding motif may have a degree of similarity to a best fit sequence alignment to β integrin (SEQ ID NO:47) of at least 40%, at least 50%, or at least 60%. In certain embodiments, the peptide has a sequence that is not identical to a best fit sequence alignment to β integrin (SEQ ID NO:47). In certain embodiments, the CRKL binding motif may have a best fit sequence alignment to a β integrin (SEQ ID NO:47) PSI domain region. The CRKL binding motif may have a best fit sequence alignment to a β integrin (SEQ ID NO:47) PSI domain region selected from the group consisting of amino acids 10 to 29; 15 to 34; 18 to 37; 36 to 55; 39 to 58; 45 to 64; 94 to 113; 196 to 215; 198 to 213; 203 to 222; 244 to 263; 330 to 349; 377 to 396; 379 to 398; 380 to 399; 398 to 417; 400 to 419; 413 to 432; 447 to 466; 460 to 479; 460 to 479; 464 to 483; 469 to 488; 474 to 493; 475 to 494; 512 to 533; 519 to 538; 551 to 570; 574 to 593; 577 to 596; 579 to 598; 590 to 609; 596 to 615; 613 to 632; 615 to 634; 616 to 635; 644 to 663; 648 to 667; 663 to 682; 674 to 693; 682 to 701; 721 to 740; 727 to 746; and 779 to 798. In certain embodiments, the CRKL binding motif has a sequence selected from the group consisting of SEQ ID NO:1 through SEQ ID NO:46. In certain embodiments, the isolated peptide of may be further defined as a cyclic peptide.

Various CRKL binding peptides may be used with the present invention. A non-limiting list of CRKL binding peptides is provided below (SEQ ID: 1-46).

TABLE 1
Tumor Homing Peptides Share Similarity with 131 Integrin

| Peptide | | Aligned | | |
|---|---|---|---|---|
| ID | Peptide | Region | Matched residue | Score |
| 1 | HTCWGARDVAQPSGTVRCLK | 10-29 | HTCWGARDVAQPSGTVRCLK | 34 |
| 2 | TSCVRTGHDENLLKAAYCSS | 15-34 | TSCVRTGHDENLLKAAYCSS | 61 |
| 3 | VACDISAVERLPASARSCKT | 18-37 | VACDISAVERLPASARSCKT | 55 |
| 4 | GPCAATGVNPGDHGAAVCDQ | 36-55 | GPCAATGVNPGDHGAAVCDQ | 42 |
| 5 | LGCNKGRYWLSTRLSVSCAL | 39-58 | LGCNKGRYWLSTRLSVSCAL | 41 |
| 6* | YRCTLNSPFFWEDMTHECHA | 45-64 | YRCTLNSPFFWEDMTHECHA | 40 |
| 7 | KLCYRSSAGSELRPPEKCAY | 94-113 | KLCYRSSAGSELRPPEKCAY | 44 |
| 8 | VRCNEAQLQDSGTVPHPCLR | 196-215 | VRCNEAQLQDSGTVPHPCLR | 36 |
| 9 | RTCEEVRNRALEELTNFCPY | 198-213 | RTCEEVRNRALEELTNFCPY | 44 |
| 10 | LRCPLEVDRPNRDPAFLCSQ | 203-222 | LRCPLEVDRPNRDPAFLCSQ | 25 |
| 11 | NRCMPGFLDDADSAASPCGS | 244-263 | NRCMPGFLDDADSAASPCGS | 53 |
| 12 | GNCMGLQVSELFMGPYKCRQ | 330-349 | GNCMGLQVSELFMGPYKCRQ | 32 |
| 13 | SRCHALRSQSVSTSAGACIS | 377-396 | SRCHALRSQSVSTSAGACIS | 43 |
| 14 | RSCVNSDTGVLQRGAPSCLF | 379-398 | RSCVNSDTGVLQRGAPSCLF | 37 |
| 15 | QHCVKGQFPFRESVTITCNS | 380-399 | QHCVKGQFPFRESVTITCNS | 47 |
| 16 | MHCTSQTLRGTPSLAPKCSD | 398-417 | MHCTSQTLRGTPSLAPKCSD | 43 |
| 17 | YSCTRLNGTGLQNPPSACDR | 400-419 | YSCTRLNGTGLQNPPSACDR | 37 |
| 18 | RGCWRDSTAWHVSYPVECLA | 413-432 | RGCWRDSTAWHVSYPVECLA | 35 |
| 19 | TLCRSLEHEVGLFKPRECPF | 447-466 | TLCRSLEHEVGLFKPRECPF | 47 |
| 20 | VVCFMERQMGTDVVSPMCVN | 460-479 | VVCFMERQMGTDVVSPMCVN | 45 |
| 21 | WVCTSASQDTRLKEPGMCIA | 460-479 | WVCTSASQDTRLKEPGMCIA | 35 |
| 22 | PNCDLDDIVLNPYTAGPCGT | 464-483 | PNCDLDDIVLNPYTAGPCGT | 35 |
| 23 | PGCVVSPFALSAQGTSVCTI | 469-488 | PGCVVSPFALSAQGTSVCTI | 47 |
| 24 | RGCTEAAGLVIGITTHQCGN | 469-488 | RGCTEAAGLVIGITTHQCGN | 49 |
| 25 | VFCCGSYCGGVEMLASRCGH | 474-493 | VFCCGSYCGGVEMLASRCGH | 40 |
| 26 | GDCETNNVTKVGGITRNCVG | 475-494 | GDCETNNVTKVGGITRNCVG | 41 |
| 27 | TTCNKSMSSQPMRDSRECHR | 514-533 | TTCNKSMSSQPMRDSRECHR | 53 |
| 28 | EGCSDIMNTAAERVTGDCSY | 519-538 | EGCSDIMNTAAERVTGDCSY | 68 |
| 29 | KICPVTNMWTTPSWAHKCGM | 551-570 | KICPVTNMWTTPSWAHKCGM | 42 |
| 30 | NNCPVEGSQQNYSGATWCRA | 574-593 | NNCPVEGSQQNYSGATWCRA | 37 |
| 31 | RTCQVRSNNISPRMALACVT | 577-596 | RTCQVRSNNISPRMALACVT | 32 |
| 32 | TECRGASSGSVSGAATDCRD | 579-598 | TECRGASSGSVSGAATDCRD | 44 |
| 33 | ARCREDTGFMGLGSANICTD | 590-609 | ARCREDTGFMGLGSANICTD | 43 |
| 34 | NDCSAHAQPGWDEVPPMCNQ | 590-609 | NDCSAHAQPGWDEVPPMCNQ | 34 |
| 35 | TLCPPASMGLGREKPRLCSV | 596-615 | TLCPPASMGLGREKPRLCSV | 32 |
| 36 | RECGRTVHRYPWGSPESCER | 613-632 | RECGRTVHRYPWGSPESCER | 44 |
| 37 | LGCMASMLREFEGATHACTQ | 615-634 | LGCMASMLREFEGATHACTQ | 39 |
| 38 | DRCVLVRPEFGRGDARLCHS | 616-635 | DRCVLVRPEFGRGDARLCHS | 32 |
| 39 | VECVMASASTDGTAAHPCKP | 644-663 | VECVMASASTDGTAAHPCKP | 53 |
| 40 | DACSRFLGERVDATAAGCSR | 648-667 | DACSRFLGERVDATAAGCSR | 39 |
| 41 | NQCSSLLTYQGWKRTKDCIP | 663-682 | NQCSSLLTYQGWKRTKDCIP | 33 |
| 42 | KSCGKYGLIVGQPFAEHCPP | 674-693 | KSCGKYGLIVGQPFAEHCPP | 34 |
| 43 | GTCPRQFFHMQEFWPSDCSR | 682-701 | GTCPRQFFHMQEFWPSDCSR | 29 |
| 44 | PNCYSGDGEISSHIPVQCLM | 721-740 | PNCYSGDGEISSHIPVQCLM | 34 |
| 45 | GYCLTVVGGAVLTIALLCVT | 727-746 | GYCLTVVGGAVLTIALLCVT | 50 |
| 46 | IGCNHPSPLGSTVVPTYCFK | 779-798 | IGCNHPSPLGSTVVPTYCFK | 35 |

### DEFINITIONS

A “targeting moiety” is a term that encompasses various types of affinity reagents that may be used to enhance the localization or binding of a substance to a particular location in an animal, including organs, tissues, particular cell types, diseased tissues or tumors. Targeting moieties may include peptides, peptide mimetics, polypeptides, antibodies, antibody-like molecules, nucleic acids, aptamers, and fragments thereof. In certain embodiments, a targeting moiety will enhance the localization of a substance to cells expressing CRKL extracellularly, i.e., CRKL being associated with the cell surface or associated with surrounding extracellular matrix. Selective binding of a targeting moiety of the present invention, e.g., a targeting peptide, as well as variants and fragments thereof is when the targeting moiety binds a target (e.g., CRKL) and does not significantly bind to unrelated proteins. A targeting moiety is still considered to selectively bind even if it also binds to other proteins that are not substantially homologous with the target so long as such proteins share homology with a fragment or domain of the peptide target of the antibody. In this case, it would be understood that target moiety binding to the target is still selective despite some degree of cross-reactivity. Typically, the degree of cross-reactivity can be determined and differentiated from binding to the target.

A “tumor targeting peptide” is a peptide comprising a CRKL binding motif, said motif defined as: being from 6 to 20 amino acids in length, having a degree of similarity to a corresponding best fit sequence alignment to β integrin (SEQ ID NO:47) of at least 25%; and wherein the targeting peptide is 100 amino acids or less in length and characterized by selective localization to an organ, tissue or cell type under physiological conditions, which includes specific binding with an extracellular CRKL. Selective localization may be determined, for example, by methods disclosed below, wherein the putative targeting peptide sequence binds to a protein that is displayed on the outer surface of a phage.

A “subject” refers generally to a mammal. In certain preferred embodiments, the subject is a mouse or rabbit. In even more preferred embodiments, the subject is a human.

### CRKL (Chicken Tumor Virus Number 10 Regulator of Kinase-Like Protein)

Chronic myelogenous leukemia (CML) is a hematologic malignancy in which uncontrolled proliferation of granulocytes occurs. It often is characterized by the reciprocal translocation of chromosomes 9 and 22, which relocates the Ableson (abl) protooncogene onto the 3′-end of the breakpoint cluster region (bcr). This produces a chimeric bcr-abl gene encoding a p210.sup.bcr-abl fusion protein, which is tumorigenic and is necessary for the growth of CML cells (Szczylik et al., 1991; Skorski et al., 1994; Tari et al., 1994; McGahon et al., 1994; Bedi et al., 1994).

The bcr-abl protein can autophosphorylate at the 177 tyrosine amino acid found within the first exon of bcr. When phosphorylated, the bcr domain of the bcr-abl protein binds to the SH2 domain of the growth factor receptor-bound protein 2 (Grb2) adaptor protein. Through the SH3 domain, Grb2 binds to the human Son of sevenless 1 (hSos1) GDP/GTP exchange factor resulting in ras protein activation. The bcr-abl protein can also transphorylate the 177 tyrosine amino acid found within the normal bcr protein. It is believed that when the normal bcr protein becomes tyrosine phosphorylated at amino acid 177, it also will complex with Grb2. When the bcr-abl protein is expressed, the p46 and p52 Shc (Puil et al., 1994) proteins become tyrosine phosphorylated as well. These Shc proteins have also been shown to form stable complexes with Grb2. Therefore, Grb2 appears to play a very important role in the tumorigenicity mediated by the bcr-abl protein (Puil et al., 1994; Pendergast et al., 1993).

Crk-like (CRKL), another adaptor protein, also has been found to bind to bcr-abl. Unlike Grb2, CRKL binds to bcr-abl through the abl domain. Through its SH3 domain, CRKL can also bind to hSos1, which again leads to Ras protein activation (ten Hoeve et al., 1994a and 1994b). Thus, via the Grb2 and CRKL adaptor proteins, the bcr-abl protein has been linked to ras activation, which is known to lead to tumorigenesis. When ras protein expression is inhibited, proliferation of CML cells is also inhibited. Therefore, one of the major pathways in which bcr-abl protein promotes CML proliferation is by activating ras protein (Skorski et al., 1994 and 1995).

The inventors found that a specific interaction between the intracellular signaling protein CRKL and a regulatory (rather than ligand-binding) β₁ integrin extracellular domain was observed. Surprisingly, the inventors found that CRKL, conventionally known as intracellular adaptors, targets the plexin-semaphorin-integrin (PSI) domain of β₁ integrin located outside of the cell, triggers MAP kinases, and promotes cell growth and survival. Without wishing to be bound by any theory, these results support the idea that an unrecognized integrin-mediated outside-in function exists for intracellular mediators, such as SH3-containing proteins, in activating the MAP kinase pathway.

### Sequence Alignment Analysis

Sequence similarity is defined as sequence identity between two nucleotide sequences, but does not necessarily indicate that two sequences have common ancestry. For example, 25% similarity means that 25 nucleotide positions out of 100 are identical in the two nucleotide sequences. “Best fit sequence alignment” in the present invention is defined as a sequence analysis by using sequence alignment methods known to those of ordinary skill in the art to find the best-matching piecewise (local) or global alignments of two or more sequences. As would be known to one of skill, various algorithms may be used for sequence comparison, e.g., BLAST alignments, etc.

Sequence alignment between tumor-homing phage peptides and β₁ integrin may be analyzed using the techniques disclosed herein or as would be. The inventors used the Peptide Match software codified in Perl 5.8.1 based on RELIC50. The program calculates similarity based on a predefined residue window size between an affinity selected peptide sequence and the target protein sequence from N- to C-protein terminus in one-residue shifts. The peptide-protein similarity scores for each residue were calculated based on a BLOSUM62 amino acid substitution matrix modified to adjust for rare amino acid representation. Thresholds may be set at least 4 identical residues between the peptide and the protein segment to discriminate significant similarities from nonspecific background matches.

### Proteins and Peptides

In certain embodiments, the present invention concerns novel compositions comprising at least one protein or peptide. As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide” are used interchangeably herein.

In certain embodiments the size of at least one protein or peptide may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues. For example, a targeting peptide may be present in a fusion protein to result in a protein.

In some aspects the size of a tumor targeting peptide defined in the present invention may comprise, but is not limited to, 5, 6, 7, 8, 9, 10, 11, 12, 13, 14, 15, 16, 17, 18, 19, 20 amino acid residues. In other aspects the size of a tumor targeting peptide may comprise, but is not limited to, 5, 6, 7, 8, 9, 10, 11, 12, 13, 14, 15, 16, 17, 18, 19, 20, 21, 22, 23, 24, 25, 26, 27, 28, 29, 30 amino acid residues, or any range derivable therein. In certain embodiments, peptides less than or equal to 20 amino acids, or peptides 6-10 amino acids in length may be used.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moiety. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid, including but not limited to Aad, 2-Aminoadipic acid; EtAsn, N-Ethylasparagine; Baad, 3-Aminoadipic acid, Hyl, Hydroxylysine; Bala, β-alanine, β-Amino-propionic acid; AHyl, allo-Hydroxylysine; Abu, 2-Aminobutyric acid; 3Hyp, 3-Hydroxyproline; 4Abu, 4-Aminobutyric acid, piperidinic acid; 4Hyp, 4-Hydroxyproline; Acp, 6-Aminocaproic acid, Ide, Isodesmosine; Ahe, 2-Aminoheptanoic acid; AIle, allo-Isoleucine; Aib, 2-Aminoisobutyric acid; MeGly, N-Methylglycine, sarcosine; Baib, 3-Aminoisobutyric acid; MeIle, N-Methylisoleucine; Apm, 2-Aminopimelic acid; MeLys, 6-N-Methyllysine; Dbu, 2,4-Diaminobutyric acid; MeVal, N-Methylvaline; Des, Desmosine; Nva, Norvaline; Dpm, 2,2′-Diaminopimelic acid; Nle, Norleucine; Dpr, 2,3-Diaminopropionic acid; Orn, Ornithine; and EtGly, N-Ethylglycine.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (world wide web at ncbi.nlm.nih.gov). The coding regions for known genes may be amplified and/or expressed using the techniques disclosed herein or as would be know to those of ordinary skill in the art. Alternatively, various commercial preparations of proteins, polypeptides and peptides are known to those of skill in the art.

### Fusion Proteins

Other embodiments of protein conjugates concern fusion proteins. These molecules generally have all or a substantial portion of a tumor targeting peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to, for example, facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the instant invention comprise an LPR targeting peptide linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually any protein or peptide could be incorporated into a fusion protein comprising a targeting peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

### Protein Purification

In certain embodiments a protein or peptide may be isolated or purified. Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. A particularly efficient method of purifying peptides is fast performance liquid chromatography (FPLC) or even high performance liquid chromatography (HPLC).

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

### Synthetic Peptides

Because of their relatively small size, the targeting peptides of the invention can be synthesized in solution or on a solid support in accordance with conventional techniques. Various automatic synthesizers are commercially available and can be used in accordance with known protocols. See, for example, Stewart and Young, 1984; Tam et al., 1983; Merrifield, 1986; Barany and Merrifield, 1979, each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

### Therapeutic or Diagnostic Conjugates

Targeting moieties identified using these methods may be coupled or attached to various substances, including therapeutic or diagnostic agents, for the selective delivery of the conjugate to a desired organ, tissue or cell type in the mouse model system. Embodiments of the invention are directed to the treatment of a disease or a disorder, preferably, a cancer. The tumor targeting peptide in the present invention may be attached to a molecule; for example, the molecule may be a protein and the peptide may be conjugated or fused to the protein to form a protein conjugate, wherein the protein conjugate is not a naturally occurring protein. The peptide may be positioned at a terminus of the protein. Said molecule may be a pro-apoptosis agent, an anti-angiogenic agent, a cytokine, a cytotoxic agent, a drug, a chemotherapeutic agent, a hormone, a growth factor, an antibiotic, an antibody or fragment or single chain thereof, a survival factor, an anti-apoptotic agent, a hormone antagonist, an antigen, a peptide, a protein, a diagnostic agent, a radioisotope, or an imaging agent.

The tumor targeting peptide may be attached to a macromolecular complex, such as a virus, a bacteriophage, a bacterium, a liposome, a microparticle, a magnetic bead, a yeast cell, or a mammalian cell. In certain embodiments, said peptide is attached to a virus, such as a lentivirus, papovavirus, adenovirus, retrovirus, AAV, vaccinia virus or herpes virus. Said peptide may be attached to a solid support, such as a microtiter dish or microchip.

### A. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

Examples of pro-apoptosis agent are gramicidin; magainin; mellitin; defensin; cecropin; (KLAKLAK)₂ (SEQ ID NO:48); (KLAKKLA)₂ (SEQ ID NO:49); (KAAKKAA)₂ (SEQ ID NO:50); (KLGKKLG)₃ (SEQ ID NO:51); Bcl-2; Bad; Bak; Bax; and Bik. In certain embodiments, said pro-apoptosis agent is (KLAKLAK)₂ (SEQ ID NO:48). SEQ ID NO:48 may consist of D amino acids.

B. Angiogenic Inhibitors

Proliferation of tumors cells relies heavily on extensive tumor vascularization, which accompanies cancer progression. Thus, inhibition of new blood vessel formation with anti-angiogenic agents and targeted destruction of existing blood vessels have been introduced as an effective and relatively non-toxic approach to tumor treatment. (Arap et al., 1998; Arap et al., 1998; Ellerby et al., 1999). A variety of anti-angiogenic agents and/or blood vessel inhibitors are known. (e.g., Folkman, 1997; Eliceiri and Cheresh, 2001).

In certain embodiments the present invention may utilize administration of targeting moieties operatively coupled to anti-angiogenic agents, such as a thrombospondin, an angiostatin, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4, minocycline, endostatin XVIII, endostatin XV, the C-terminal hemopexin domain of matrix metalloproteinase-2, the kringle 5 domain of human plasminogen, a fusion protein of endostatin and angiostatin, a fusion protein of endostatin and the kringle 5 domain of human plasminogen, the monokine-induced by interferon-gamma (Mig), a fusion protein of Mig and IP10, soluble FLT-1 (fins-like tyrosine kinase 1 receptor), or kinase insert domain receptor (KDR). Said molecule may be a cytokine selected from the group consisting of interleukin 1 (IL-1), IL-2, IL-5, IL-10, IL-11, IL-12, IL-18, interferon-γ (IF-γ), IF-α, IF-β, a tumor necrosis factor, or GM-CSF (granulocyte macrophage colony stimulating factor).

C. Cytotoxic Agents

Chemotherapeutic (cytotoxic) agents may be coupled to a targeting peptide of the present invention and used to treat various hyperproliferative or neoplastic disease states, including cancer. Chemotherapeutic (cytotoxic) agents of potential use include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences” 15^{th} ed., pp 1035-1038 and 1570-1580, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Of course, all dosages and agents described herein are exemplary rather than limiting, and other doses or agents may be used by a skilled artisan for a specific patient or application. Any dosage in-between these points, or range derivable therein is also expected to be of use in the invention.

D. Alkylating Agents

Alkylating agents are drugs that directly interact with genomic DNA to prevent cells from proliferating. This category of chemotherapeutic drugs represents agents that affect all phases of the cell cycle, that is, they are not phase-specific. An alkylating agent, may include, but is not limited to, a nitrogen mustard, an ethylenimene, a methylmelamine, an alkyl sulfonate, a nitrosourea or a triazines. They include but are not limited to: busulfan, chlorambucil, cisplatin, cyclophosphamide (cytoxan), dacarbazine, ifosfamide, mechlorethamine (mustargen), and melphalan.

E. Antimetabolites

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

F. Natural Products

Natural products generally refer to compounds originally isolated from a natural source, and identified as having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

G. Antibiotics

Certain antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of cytotoxic antibiotics include, but are not limited to, bleomycin, dactinomycin, daunorubicin, doxorubicin (Adriamycin), plicamycin (mithramycin) and idarubicin.

H. Miscellaneous Cytotoxic Agents

Miscellaneous cytotoxic agents that do not fall into the previous categories include, but are not limited to, platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are not limiting and it is contemplated that any known cytotoxic, cytostatic or cytocidal agent may be attached to targeting peptides and administered to a targeted organ, tissue or cell type within the scope of the invention.

I. Imaging Agents and Radioisotopes

In certain embodiments, the targeting moieties of the present invention may be attached to imaging agents of use for imaging and diagnosis of various diseased organs, tissues or cell types. Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled proteins or peptides of the present invention may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides according to the invention may be labeled with technetium-^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups that are often used to bind radioisotopes that exist as metallic ions to peptides are diethylenetriaminepenta-acetic acid (DTPA) and ethylene diaminetetra-acetic acid (EDTA). Also contemplated for use are fluorescent labels, including rhodamine, fluorescein isothiocyanate and renographin.

In certain embodiments, the claimed proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

In still further embodiments, a targeting moiety may be operatively coupled to a nanoparticle. Nanoparticles include, but are not limited to colloidal gold and silver nanoparticles. Metal nanoparticles exhibit colors in the visible spectral region. It is believed that these colors are the result of excitation of surface plasmon resonances in the metal particles and are extremely sensitive to particles' sizes, shapes, and aggregation state; dielectric properties of the surrounding medium; adsorption of ions on the surface of the particles (For examples see U.S. Patent Application 20040023415, which is incorporated herein by reference).

J. Cross-Linkers

Cross linkers may also be included in a fusion protein or other construct comprising a CRKL targeting peptide; for example, cross linkers may be useful for conjugating a targeting peptide to a liposome or a therapeutic compound. Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. Nos. 5,603,872 and 5,401,511, each specifically incorporated herein by reference in its entirety. Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites are dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl)carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups.

### Nucleic Acids

Nucleic acids according to the present invention may encode a targeting peptide, a targeting antibody, a therapeutic polypeptide a fusion protein or other protein or peptide. The nucleic acid may be derived from genomic DNA, complementary DNA (cDNA) or synthetic DNA.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of almost any size, determined in part by the length of the encoded protein or peptide.

It is contemplated that targeting peptides and fusion proteins may be encoded by any nucleic acid sequence that encodes the appropriate amino acid sequence. The design and production of nucleic acids encoding a desired amino acid sequence is well known to those of skill in the art, using standardized codon tables. In preferred embodiments, the codons selected for encoding each amino acid may be modified to optimize expression of the nucleic acid in the host cell of interest.

### Targeted Delivery of Gene Therapy Vectors

Targeting peptides may, in certain embodiments, be coupled to a gene therapy vector to selectively or preferentially target cells expressing CRKL on the cell surface, such as certain tumor cells. There are a number of ways in which gene therapy vectors may be introduced into cells. In certain embodiments of the invention, the gene therapy vector comprises a virus. The ability of certain viruses to enter cells via receptor-mediated endocytosis, to integrate into host cell genome or be maintained episomally, and express viral genes stably and efficiently have made them attractive candidates for the transfer of foreign genes into mammalian cells (Ridgeway, 1988; Nicolas and Rubinstein, 1988.; Baichwal and Sugden, 1986; Temin, 1986). Preferred gene therapy vectors are generally viral vectors. DNA viruses used as gene therapy vectors include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986).

One of the preferred methods for in vivo delivery involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include, but is not limited to, constructs containing adenovirus sequences sufficient to (a) support packaging of the construct and (b) to express an antisense or a sense polynucleotide that has been cloned therein.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic innoculation into the brain (Le Gal La Salle et al., 1993).

In preferred embodiments, certain advantages may be gained from coupling therapeutic molecules or substances to tumor targeting peptides that target to cells expressing and bearing CRKL on the surface, such as tumor cells. Specifically, moieties that home to tumor vasculature have been coupled to cytotoxic drugs or proapoptotic peptides to yield compounds were more effective and less toxic than the parental compounds in experimental models of mice bearing tumor xenografts (Arap et al., 1998; Ellerby et al, 1999). The insertion of the RGD-4C peptide into a surface protein of an adenovirus has produced an adenoviral vector that may be used for tumor targeted gene therapy (Arap et al., 1998).

The peptide motif allows for cell targeting, for instance, by comprising a targeting moiety of the invention, and/or a ligand for a cell surface binding site. The peptide motif optionally can comprise other elements of use in cell targeting (e.g., a single-chain antibody sequence). The peptide binding motif may be generated by the insertion, and may comprise, for instance, native and normative sequences, or may be entirely made up of normative sequences. The peptide motif that results from the insertion of the normative amino acid sequence into the chimeric fiber protein can be either a high affinity peptide (i.e., one that binds its cognate binding site, e.g., CRKL, when provided at a relatively low concentration) or a low affinity peptide (i.e., one that binds its cognate binding site, e.g., CRKL, when provided at a relatively high concentration). Preferably, however, the resultant peptide motif is a high affinity motif, particularly one that has a high affinity for its cognate binding site due to its constraint within the adenovirus fiber protein.

Other gene transfer vectors may be constructed from retroviruses. (Coffin, 1990.) In order to construct a retroviral vector, a nucleic acid encoding protein of interest is inserted into the viral genome in the place of certain viral sequences to produce a virus that is replication-defective. In order to produce virions, a packaging cell line containing the gag, pol, and env genes, but without the LTR and packaging components, is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., 1975).

Other viral vectors may be employed as targeted gene therapy vectors. Vectors derived from viruses such as vaccinia virus (Ridgeway, 1988; Baichwal and Sugden, 1986), adeno-associated virus (AAV) (Ridgeway, 1988; Baichwal and Sugden, 1986; Hermonat and Muzycska, 1984), and herpes viruses may be employed.

In a further embodiment of the invention, gene therapy construct may be entrapped in a liposome. Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa, and hepatoma cells. Nicolau et al., (1987.) accomplished successful liposome-mediated gene transfer in rats after intravenous injection.

Gene therapy vectors of the invention may comprise various transgenes, which are typically encoded DNA or RNA of an expression vector. Gene therapy may be used for the expression of a therapeutic gene, expression of VEGFR-1/NRP-1 to enhance neo-vascularization or for the inhibition of VEGFR-1/NRP-1 expression for the treatment of disease states associated with neo-vascularization. DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 21-25 base pairs and having a nucleotide sequence identical or nearly identical to an expressed target gene or RNA within the cell. Interference may result in suppression of expression. In addition, DNA and RNA may be single, double, triple, or quadruple stranded.

Pharmaceutical Compositions

Pharmaceutical compositions of the present invention comprise an effective amount of one or more said tumor targeting peptide or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one tumor targeting peptide in the present invention or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington: The Science and Practice of Pharmacy, 21th Ed. Mack Printing Company, 2005, incorporated herein by reference).

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

### Combination Therapies

In order to increase the effectiveness of an isolated tumor targeting peptide containing a CRKL binding motif, it may be desirable to combine these compositions with other agents or therapy methods, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that the tumor targeting peptide could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, the tumor targeting peptide is “A” and the secondary agent, such as radio- or chemotherapy, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with a therapy with the tumor targeting peptide. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

d. Genes

In yet another embodiment, the secondary treatment is a gene therapy in which a therapeutic polynucleotide is administered before, after, or at the same time a first therapeutic agent comprising an isolated tumor targeting peptide containing a CRKL binding motif. Delivery of the therapeutic agent in conjunction with a second vector encoding one of the following gene products will have a combined anti-hyperproliferative effect on target tissues.

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Selection of Tumor-Homing Peptides In Vivo

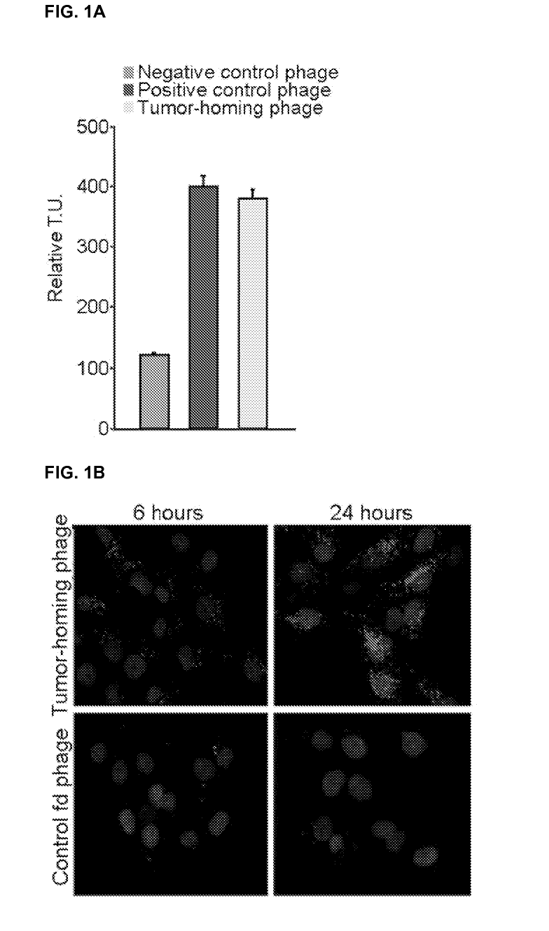

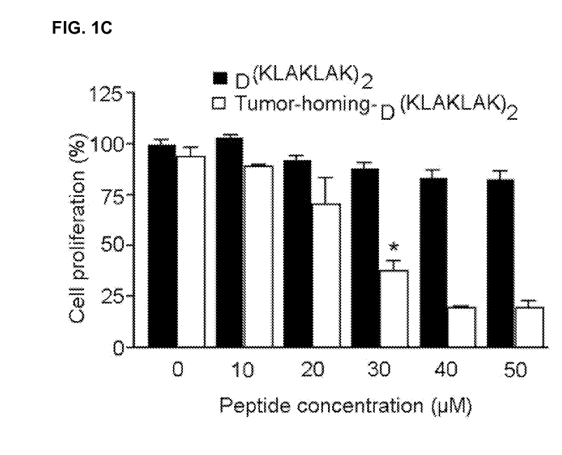

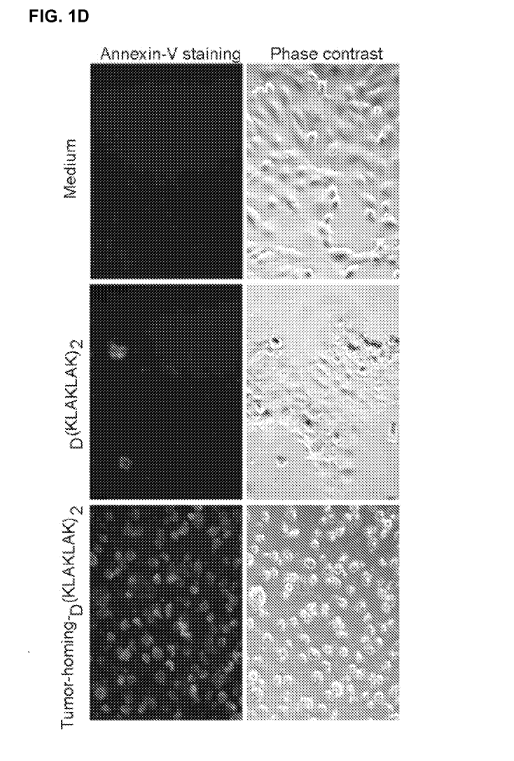

A phage display random peptide library (Arap et al., 1998; Pasqualini and Ruoslahti, 1996; Pasqualini et al., 2001) was administered intravenously into nu/nu (nude) mice bearing human DU145-derived prostate cancer xenografts and the tumors were recovered after a 24 hour circulation timeframe. After three rounds of selection, an enriched population of tumor-targeting phage was recovered and the DNA corresponding to the peptide inserts displayed by individual phage clones was sequenced (Table 1). The dominant peptide was selected, amino acid sequence YRCTLNSPFFWEDMTHECHA (SEQ ID NO:1) for functional characterization. First, the tumor-targeting specificity of the YRCTLNSPFFWEDMTHECHA (SEQ ID NO:1) peptide was evaluated in vivo in DU145-derived tumor-bearing mice. After systemic intravenous administration of the individual YRCTLNSPFFWEDMTHECHA (SEQ ID NO:1)-displaying phage clone, marked homing to tumor xenografts was observed (insertless phage served as a negative control) with no or barely detectable phage localization noted in several control organs. Consistently, DU145 prostate cancer cells were also targeted in vitro by using an aqueous to organic phase separation assay (Giordano et al., 2001) and a phage-based immunofluorescence assay (KS1767 cells) and it was found that phage displaying the peptide YRCTLNSPFFWEDMTHECHA (SEQ ID NO:1) binds to tumor cell surfaces much more than a negative control phage displaying no peptide insert (FIG. 1A, B).

TABLE 1
Tumor homing peptides (n = 75)

| Sequence | Frequency |
|---|---|
| YRCTLNSPFFWEDMTHECHA | 14.7% |
| (SEQ ID NO: 1) | |
| LGCMASMLREFEGATHACTQ | 10.7% |
| (SEQ ID NO: 2) | |
| RGCTEAAGLVIGITTHQCGN | 4.1% |
| (SEQ ID NO: 3) | |
| IGCNHPSPLGSTVVPTYCFK | 4.1% |
| (SEQ ID NO: 4) | |
| GTCPRQFFHMQEFWPSDCSR | 4.1% |
| (SEQ ID NO: 5) | |
| DRCVLVRPEFGRGDARLCHS | 2.8% |
| (SEQ ID NO: 6) | |
| EGCSDIMNTAAERVTGDCSY | 2.8% |
| (SEQ ID NO: 7) | |
| VFCCGSYCGGVEMLASRCGH | 2.8% |
| (SEQ ID NO: 8) | |
| RECGRTVHRYPWGSPESCER | 2.8% |
| (SEQ ID NO: 9) | |
| DACSRFLGERVDATAAGCSR | 2.8% |
| (SEQ ID NO: 10) | |
| GNCMGLQVSELFMGPYKCRQ | 2.8% |
| (SEQ ID NO: 11) | |
| Other | 45.5% |

Next, the internalization capability of the selected peptide was evaluated. To that end, a proapoptotic peptide that specifically targets eukaryotic mitochondrial membranes (Arap et al., 2004; Javadpour et al., 1996; Ellerby et al., 1999; Kolonin et al., 2004; Zurita et al., 2004) was fused to the tumor-homing peptide. Targeted cell killing relative to controls (FIG. 1C), indicated that the peptide YRCTLNSPFFWEDMTHECHA (SEQ ID NO:1) mediates ligand-directed internalization. Programmed cell death was confirmed by an annexin-V staining assay (FIG. 1D). Of note, selective targeting and internalization of pro-apoptotic peptides offers possibilities for the design of modular targeted peptidomimetic-based anti-tumor therapy (Arap et al., 2004; Ellerby et al., 1999; Kolonin et al., 2004; Zurita et al., 2004) Together, these results show that the peptide YRCTLNSPFFWEDMTHECHA (SEQ ID NO:1) targets tumor cells and enables internalization.

### Reagents

All cell lines were from the American Tissue Type Collection (ATCC). The following antibodies were used in studies described herein: anti-CRKL (Santa Cruz, Cell Signaling, Epitomics or Upstate Biotechnology), anti-phospho-CRKL (Cell Signaling), anti-β₁ integrin (Chemicon or BD Transduction Laboratories), anti-IL11R (Santa Cruz), anti-β₃ and anti-β₅ integrins47, anti-EGFR48, anti-grb2 (Santa Cruz), anti-alpha6 integrin (Chemicon), anti-AHSG/Feutin A (R&D Systems), pre-immune serum (Jackson Laboratory), anti-FAK (Upstate), anti-Histone H1 (Santa Cruz), anti-phospho-paxillin (Cell Signaling), anti-phospho-130Cas (Santa Cruz), anti-phospho-Erk1/2 (Cell Signaling or Biosource), anti-phospho-Elk-1 (Cell Signaling), anti-His (Santa Cruz), anti-gst (Santa Cruz), and anti-GAPDH (Ambion). Peptides were synthesized and cyclized to our specifications (AnaSpec). Six-week-old male nude mice were commercially obtained (Harlen) and tumor xenografts were generated as described (Arap et al., 2004; Marchio et al., 2004). The Institutional Animal Care and Use Committee (IACUC) at the University of Texas M. D. Anderson Cancer Center (UTMDACC) reviewed and approved all experimental procedures.

### Phage Display Random Peptide Library Selection

In vivo phage screenings were performed as described (Arap et al., 1998; Arap et al., 2004; Pasqualini and Ruoslahti, 1996; Pasqualini et al., 2001). A random phage library displaying an insert with the general arrangement X₂CX₁₂CX₂ (C, cysteine; X, any residue) was systemically administered (tail vein) into athymic nude mice bearing tumor xenografts derived from human DU145 prostate cancer cells and allowed to circulate for 24 hours. Mice were placed under deep anesthesia, tumor xenografts excised, weighed, and the bound phage population was recovered and processed (Arap et al., 1998; Arap et al., 2004; Pasqualini and Ruoslahti, 1996; Pasqualini et al., 2001). Three serial rounds of in vivo selection were performed.

### Example 2

### A Tumor-Homing Peptide Sequence Mimics a Regulatory Integrin Extracellular Domain

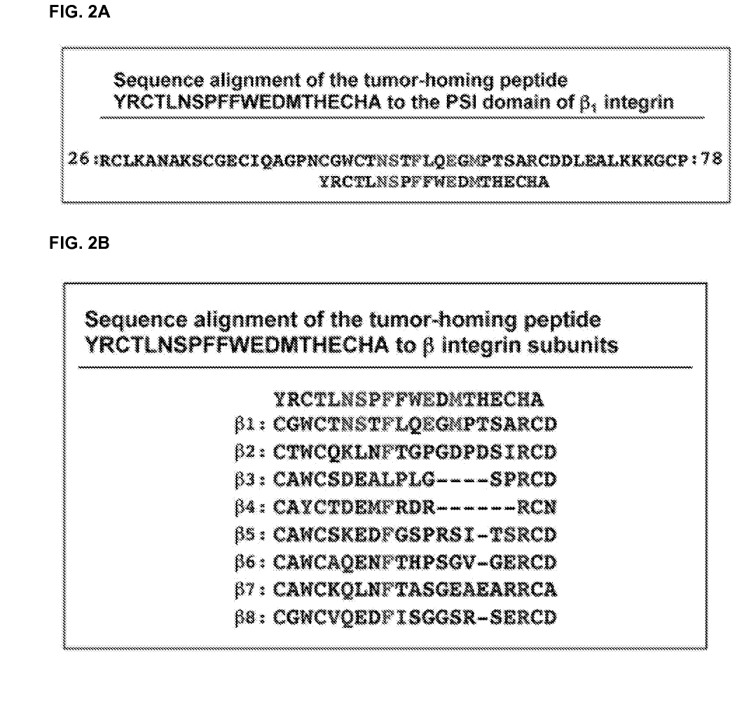

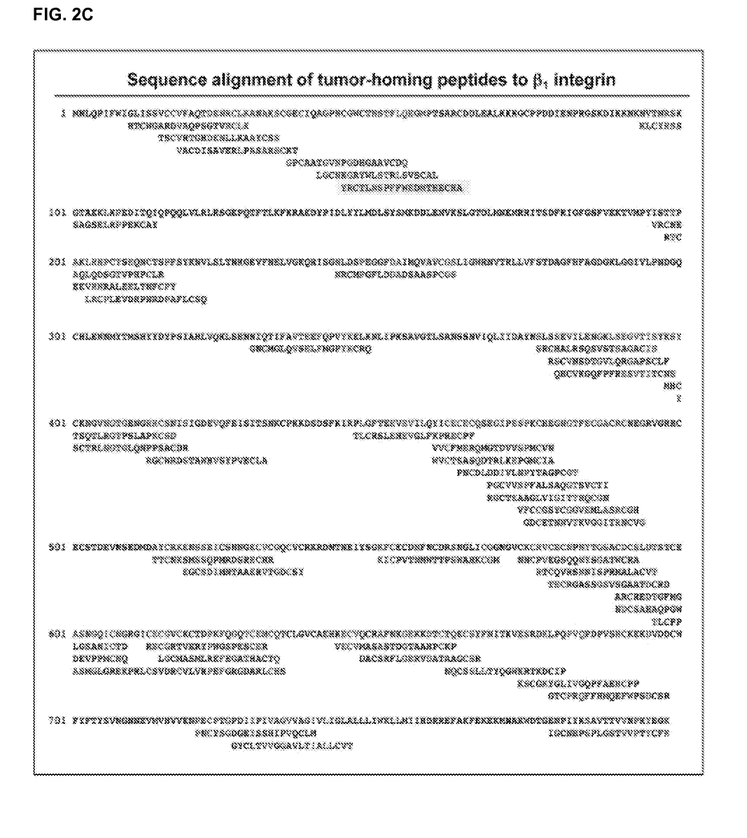

In order to determine whether the peptide sequence identified mimics a native protein, a similarity search of YRCTLNSPFFWEDMTHECHA (SEQ ID NO:1) and of the other selected peptide sequences was performed. By using standard blast search against on-line databases followed by protein sequence alignment, it was found that all unique phage-displayed peptides resembled sequences present on β₁ integrin. Unexpectedly, the dominant peptide sequence YRCTLNSPFFWEDMTHECHA (SEQ ID NO:1) had similarity to the plexin-semaphorin-integrin (PSI) extracellular domain (residues 26-78) of the β₁ integrin chain (FIG. 2A; moreover, it was found that other selected peptides also appeared within the same region (FIG. 2C). It was then determined whether the similarity of the selected peptide sequence YRCTLNSPFFWEDMTHECHA (SEQ ID NO:1) was specific for the PSI domain of the β₁ integrin sequence or common to other known integrin β chains. After fit analysis, and molecular modeling, it was concluded that the sequence identity between YRCTLNSPFFWEDMTHECHA (SEQ ID NO:1) and the PSI domain of β₁ integrin was indeed the best alignment (FIG. 2B).

### Sequence Alignment Analysis

Sequence alignment between tumor-homing phage peptides and β₁ integrin analyzed by using the Peptide Match software codified in Perl 5.8.1 based on RELIC50. The program calculates similarity based on a predefined residue window size between an affinity selected peptide sequence and the target protein sequence from N- to C-protein terminus in one-residue shifts. The peptide-protein similarity scores for each residue were calculated based on a BLOSUM62 amino acid substitution matrix modified to adjust for rare amino acid representation. Thresholds were set at least 4 identical residues between the peptide and the protein segment to discriminate significant similarities from nonspecific background matches.

### Example 3

### A Cytoplasmic Adapter Protein Serves as a Receptor for the PSI Domain-Like Tumor-Homing Peptide

The integrin PSI domain has been well characterized for its regulatory activity (Shi et al., 2005; Mould et al., 2005; Arnaout et al., 2005; Juliano et al., 2004). Given the selection results, it seems that the PSI domain might also function as a ligand-receptor binding site within β₁ integrins. In view of this, affinity chromatography was used to identify binding partners to the tumor-homing peptide YRCTLNSPFFWEDMTHECHA (SEQ ID NO:1). First a DU145-derived cell extract was pre-cleared through a control peptide column and then the pre-cleared extract was passed through the tumor-homing YRCTLNSPFFWEDMTHECHA (SEQ ID NO:1) peptide column followed by an acidic elution. A specific gel band corresponding to an ˜40-KDa protein was detected that eluted from the column.

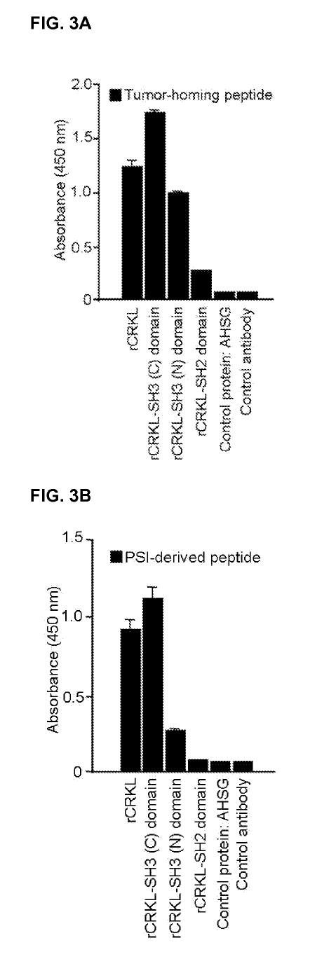

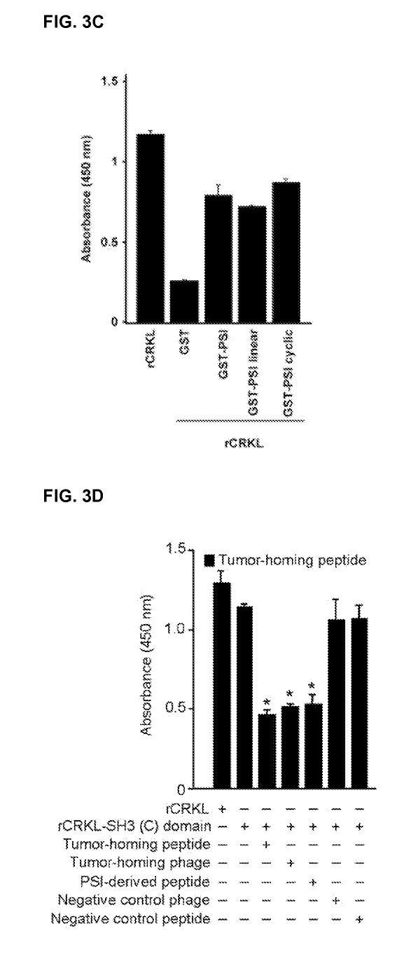

Mass spectrometry and database analysis revealed the identity of the gel band protein to be the chicken tumor virus number 10 regulator of kinase-like protein accession # NP005198 (SEQ ID NO:22) (ten Hoeve et al., 1993) (termed CRKL). These results were validated by immunoblotting the purified protein by with an anti-CRKL antibody. Next, a recombinant His-tag fusion proteins of CRKL (rCRKL) and its three corresponding domains rCRKL-SH2, rCRKL-SH3 (N), and rCRKL-SH3 (C) were designed and constructed for binding assays. It was found that the peptide YRCTLNSPFFWEDMTHECHA (SEQ ID NO:1) bound to rCRKL preferentially through the two SH3 domains of the protein; in contrast, little to no binding was detected through the SH2 domain or to control proteins (FIG. 3A). Several phage clones displaying unrelated peptide sequences showed no binding when tested under similar experimental conditions. Binding studies were also performed with a synthetic peptide directly derived from the native PSI domain of β₁ integrin (sequence NSTFLQEGMPTSA (SEQ ID NO:23); residues 50 to 62), which overlaps with the phage display-selected peptide sequences within the native PSI region (FIG. 2A,C). Again, the β₁ integrin PSI-derived peptide NSTFLQEGMPTSA (SEQ ID NO:23) bound to rCRKL, rCRKL-SH3 (N) domain, and rCRKL-SH3 (C) domain (FIG. 2B). Moreover, cyclic and linear peptide sequences from the PSI domain were generated and it was found that the cyclic disulfide bonds present in the PSI domain are not essential for the binding to CRKL (FIG. 3C). Finally, it was shown that the interaction between the SH3 (C) domain and the tumor-homing peptide is specifically inhibited by the corresponding synthetic peptides and by the phage clone itself (FIG. 3D). These findings show that the tumor-homing peptide and β₁ integrin-specific PSI domain-mimic, YRCTLNSPFFWEDMTHECHA (SEQ ID NO:1), targets the SH3 domains of CRKL.

### Affinity Chromatography and Mass Spectrometry.

Standard peptide affinity columns were made by EDC and DADPA immobilization resin (Pierce). DU145 tumor cell extracts were prepared and first passed through a non-specific control peptide column followed by the tumor-homing peptide column. Columns were washed extensively, then eluted with glycine (pH 2.2), and analyzed by SDS-PAGE. Then, the gels were Coomassie-stained. A band of ˜40 KDa was detected and excised for protein sequencing by mass spectrometry at the UTMDACC Proteomic Core Facility. The protein was identified as CRKL. Affinity purification of CRKL from serum-free condition medium was performed and confirmed by using recombinant gst-tag fusion protein expressing either the tumor-homing peptide or a mutant control peptide (Pro→Ala). Approximately 200 ml of serum-free condition medium (48 hours of culturing) was concentrated for the affinity purification. The recombinant fusion proteins were coupled to gst-resin beads and loaded on a column. The concentrated serum-free condition medium was added to the coupled columns and incubated overnight. After several washes the bound CRKL was eluted for Western blotting. The blot was probed with anti-gst and anti-CRKL antibodies.

### Phage Binding and Protein-Protein Assays

Phage binding assays on purified proteins were carried out as described (Giordano et al. 2001). Recombinant proteins were coated on microtiter wells as previous described (Cardo-Vila et al., 2003; Smith and Scott, 1993). Briefly, proteins were immobilized on microtiter wells overnight at 4° C. 50 μl of 1 μg/ml in PBS. Wells were washed twice with PBS, blocked with PBS containing 3% BSA for 2 hours at room temperature, and incubated with 10⁹ T.U. wild-type tumor-homing phage (YRCTLNSPFFWEDMTHECHA; SEQ ID NO:1), scrambled phage (YRFCTSPFHEWHLENTDMCA (SEQ ID NO:26), YRECTDSPHEFHLWNTMCAF (SEQ ID NO:27), YRCETDSPHEFHLWNTMCAF (SEQ ID NO:29), YRCETDSPHEFHLWNTFCAM (SEQ ID NO:30)), mutant phage (YRCTLNSAFFWEDMTHECHA (SEQ ID NO:25), YRCTLNSPAAAEDMTHECHA (SEQ ID NO:28)), PSI-derived cyclic phage (CNSTFLQEGMPTSAC; SEQ ID NO:23) or fd-tet phage in 50 μl of PBS containing 1.5% BSA. After 1 hour at room temperature, wells were washed ten times with PBS and phage were recovered by bacterial infection. ELISA with polyclonal anti-CKRL confirmed the presence and concentration of the CKRL recombinant proteins on the wells. To test the binding of the tumor-homing phage to SH3 containing proteins, microtiter-wells were coated at 4° C. overnight with 250 μg/ml of recombinant gst-SH3 domains (CKRL-D1, CKRL-D2, grb2-D1, grb2-D2, Lyn, src; Pronomics), rCKRL protein, and negative controls gst or BSA. For the SH3 (C) mutant binding studies, 2 μg/ml of His-tag recombinant wild-type SH3-C and mutant SH3 (C) domains were coated. The tumor-homing phage (10¹⁰ T.U.) or fd-tet phage (inserless) was added to each well and binding assay was preformed as described above. Protein-protein interaction experiment between CRKL and integrin β₁ was done on wells coated with α5β1, αvβ3 or αvβ5 integrins (Chemicon) at 1 μg/well. To evaluate the inhibitory binding of CKRL to the β₁ integrin by gst-PSI, CKRL and increasing concentration of gst-PSI or gst were pre-incubated for 15 min at room temperature prior to being added to the coated wells. After 3 hours, binding of CKRL to integrins was detected by using an anti-CKRL antibody followed by an HRP-conjugated anti-rabbit IgG. To confirm that equal amounts of the integrins were bound to the plates, parallel experiments were performed using a 1:1500 dilution of an anti-integrin antibody (Amersham Pharmacia).

### Design and Construction of Scrambled and Mutant Tumor-Homing Phage

To generate phage clones to study the binding properties to CRKL protein, phage displaying scrambled peptide sequences and mutants designed and constructed (Pro→Ala and Phe-Phe-Trp→Ala-Ala-Ala) from the selected CRKL binding tumor homing phage peptide YRCTLNSPFFWEDMTHECHA (SEQ ID NO:1). Scrambled peptide sequences (YRFCTSPFHEWHLENTDMCA (SEQ ID NO:26), YRECTDSPHEFHLWNTMCAF (SEQ ID NO:27), YRCETDSPHEFHLWNTMCAF (SEQ ID NO:29), YRCETDSPHEFHLWNTFCAM (SEQ ID NO:30) mutants (YRCTLNSAFFWEDMTHECHA (SEQ ID NO:25) and YRCTLNSPAAAEDMTHECHA (SEQ ID NO:28)) or native PSI-derived phage (CNSTFLQEGMPTSAC; SEQ ID NO:23) were cloned into the SfiI-digested fUSE5 vectors (Smith and Scott, 1993). Briefly, 500 ηg of each of the synthetic oligonucleotide templates corresponding to the displayed peptides (Sigma-Genosys) were converted to double-stranded DNA by PCR amplification with the primer set 5′ GTGAGCCGGCTGCCC 3′ (SEQ ID NO:68) and 5′ TTCGGCCCCAGCGGC 3′ (SEQ ID NO:69) (Sigma-Genosys) and 2.5 U of Taq-DNA polymerase (Promega) in 20 μl as follows: 94° C. for 2 min, followed by 35 cycles at 94° C. for 30 s, 60° C. for 30 s, and 72° C. for 30 s, followed by 72° C. for 5 min. Double-stranded DNA sequences that contained BglI restriction sites in the insert-flanking regions were purified by using a QIAquick nucleotide removal kit (Qiagen) and eluted. Oligonucleotides were digested with BglI for 2 hour at 37° C., re-purified, and ligated into SfiI-digested fUSE5 vector. The phage clones generated were PCR amplified to verify the correct insertion and nucleotide sequence. The individual phage clones were tested in phage binding assays.

### Peptide Binding and Internalization Assays

Synthetic tumor-homing peptide (YRCTLNSPFFWEDMTHECHA; SEQ ID NO:1) or PSI-derived peptide (NSTFLQEGMPTSA; SEQ ID NO:23) were coated on microtiter plates followed by blocking, and washing. The following recombinant His-tag proteins were added to the coated plates: rCRKL, rCRKL-SH2 domain, rCRKL-SH3 (N) domain, and rCRKL-SH3 (C) domain. An unrelated control protein (α2-Heremans-Schmid glycoprotein; AHSG) served as a negative control. The mixtures were incubated, washed, and labeled with the appropriate antibodies. Secondary antibodies conjugated to horse-radish peroxidase (HRP) were added followed by adding TMB substrate (Calbiochem), and analyzed by an automated ELISA reader (Bio-Tek). To determine the inhibitory activity of the tumor-homing peptide, the PSI-derived peptide, or the phage clone displaying the corresponding tumor-homing peptide were incubated with the rCRKL-SH3 (C) domain. Unrelated cyclic peptide sequences and insertless phage (fd-tet) served as negative controls. The admixtures were incubated and added to wells coated with the tumor-homing peptide. After incubation, the wells were washed, labeled with the appropriate antibodies, and processed as above.

The internalization capability of the tumor-homing peptide fused through a glycinylglycine bridge to a pro-apoptotic sequence was tested as described Arap et al., 2004; Ellerby et al., 1999; Kolonin et al., 2004; Zurita et al., 2004). The conjugated tumor-homing peptide, YRCTLNSPFFWEDMTHECHAGG (SEQ ID NO:67)-_{D}(KLAKLAK)₂ or the untargeted control peptide _{D}(KLAKLAK)₂ were synthesized and increasing equimolar peptide concentrations were added to the DU145 cells. Cell viability was assayed by the WST-1 reagent (Roche) and annexin-V staining for apoptosis (Zurita et al., 2004; Cardo-Vila et al., 2003). For tumor-homing phage localization studies, cells were incubated with 10⁹ T.U. tumor-homing (YRCTLNSPFFWEDMTHECHA; SEQ ID NO:1) or a negative control (fd-tet) phage for 6 and 24 hours. Wells were washed with 20 mM glycine to remove non-specific cell surface bound phage and then fixed with 4% paraformaldehyde. The non-permeabilized cells were incubated with rabbit anti-fd bacteriophage antibody (Sigma) for 2 hours at room temperature followed by 1 hour incubation with Cy3-labeled anti-rabbit IgG antibody (Jackson ImmunoResearch). Cells were again fixed with 4% paraformaldehyde and mounted in the presence of DAPI (Vector Laboratories) and images were acquired with an Olympus fluorescence microscope.

### Example 4

### Characterizing the Interaction with CRKL

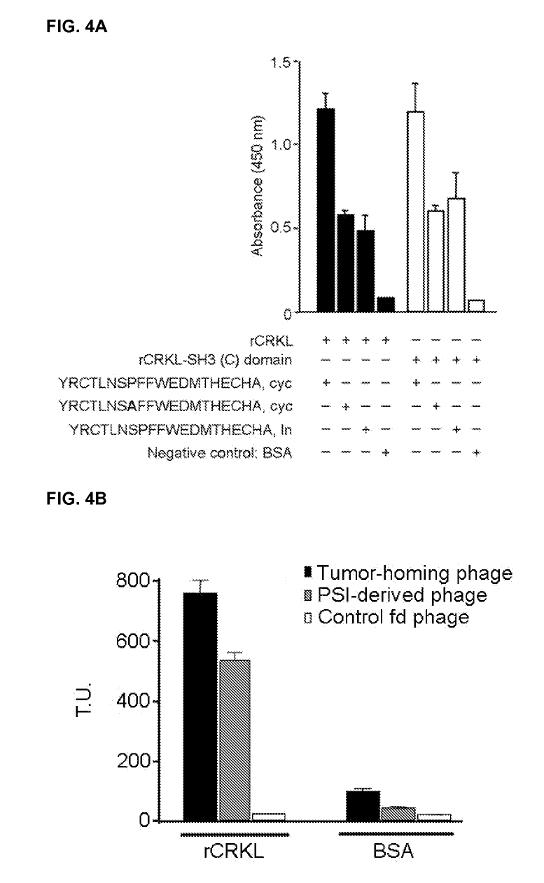

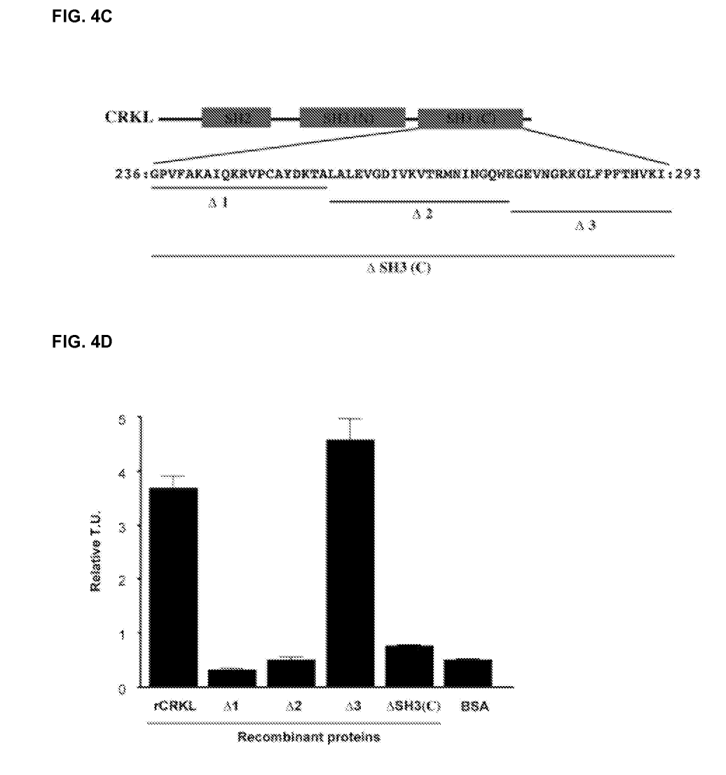

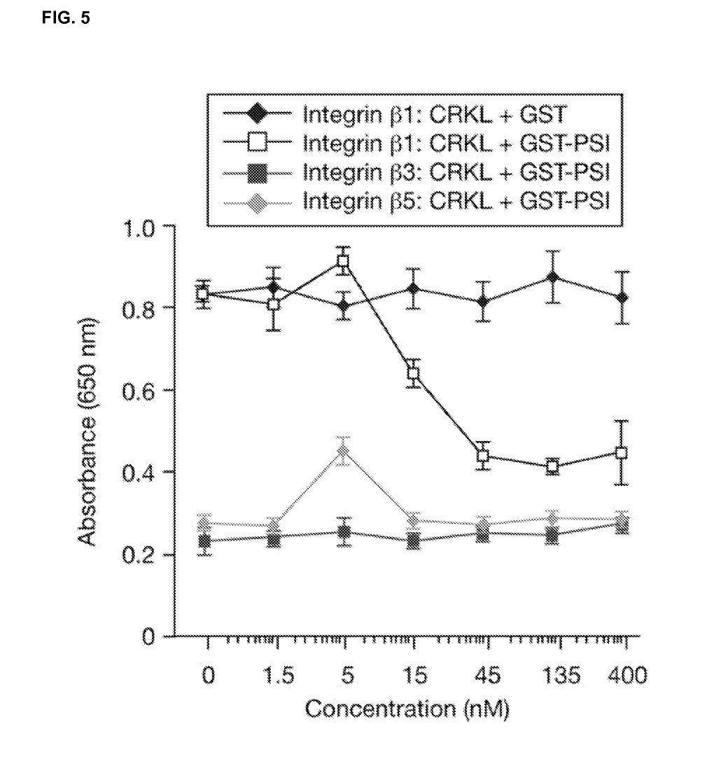

Other studies have shown that SH3 domains bind to the MOO and non-PXXP motifs in addition to motifs containing a single Pro residue (Mayer, 2001; Sicheri et al., 1997; Kang et al., 2000; Kato et al., 2000; Xu et al., 1997). Since the sequence set forth in SEQ ID NO:1 does not contain any such known motifs, site-directed mutagenesis was used to evaluate the binding attributes of the tumor-homing peptide. Results of these studies showed that the binding to the CRKL-SH3 (C) domain is dependent on the Pro residue and on the cyclic Cys-Cys disulfide bridge present in the tumor-homing peptide (FIG. 4A); fd-tet insertless phage and unrelated synthetic cyclic peptides served as negative controls. Mutational analysis of the CRKL SH3 (C) domain was also performed and results showed the binding region to be between residues Gly₂₃₆ and Trp₂₇₇ (FIG. 4C,D). In addition, it was shown that both the tumor-homing phage (SEQ ID NO:1) and the PSI-derived phage (SEQ ID NO:23) bind to recombinant CRKL (FIG. 4B). Furthermore, by using affinity chromatography, it was established that CRKL can be purified from DU145 serum-free condition medium, and that a control column prepared with a mutant form of the tumor-homing peptide no longer binds to CRKL at detectable levels under identical experimental conditions. By using reciprocal co-immunoprecipitation assays with membrane fractions, it was established that CRKL and β₁ integrin form a cell surface complex; in contrast, control antibodies raised against unrelated transmembrane receptors including anti-IL11 receptor, anti-EGF receptor, or other integrins (anti-β₃ and anti-β₅) showed no association with CRKL or β₁ integrin. Finally, it was demonstrate that protein-protein interaction between CRKL and β₁ integrin can be inhibited in a dose-dependent manner by the PSI domain expressed as a recombinant protein (IC₅₀=20 ηm); in agreement with the co-immunoprecipitation studies, control integrins showed no binding (FIG. 5).

### Mutant Constructs

All primer sets are summarized in (Table 2). The open reading frame of full-length CRKL cDNA (Invitrogen) was PCR-amplified and cloned in a pET28a (Novagen) expression vector. cDNAs corresponding to each of the SH domains were PCR-amplified and cloned into the Sac I and Xho I restriction sites of the vector. All constructs were verified by restriction digestion and DNA sequencing, transformed into the BL21 (Stragene) bacteria strain and recombinant proteins were purified on His-tag columns (Qiagen). Purified recombinant proteins were verified through Coomassie staining and Western blot analysis by using anti-CRKL and anti-His antibodies.

CRKL mutants in the SH3 domain (carboxyl terminal) were generated by PCR mutagenesis. Primer sets were designed to remove 60 bp (Δ1 and Δ2) and 54 bp (Δ3) and maintain the reading frame. For the PSI domain constructs, cyclic and linear peptide forms of the PSI domain were produced. After annealing the PSI oligonucleotides (Table 2), the double strand oligonucleotides were purified after digestion with EcoRI by using the nucleotide removal kit (QIAGEN) and cloned in pGEX4T-1 vector. To show that the CRKL protein oligomerizes, increasing concentrations of recombinant gst-CRKL were incubated with immobilized CRKL protein (His-tag recombinant form) or immobilized BSA overnight at 4° C. followed by three washes. Protein-protein interactions were detected by using an antibody against gst.

TABLE 2
PCR primers (SEQ ID NOs: 32-53)

Full-length CRKL:
5′-CACAGAGCTCAACACCATGTCCTCCGCCAGGTTCG-3′
5′-CACACTCGAGCTCGTTTTCATCTGGGTTTTGAGGG-3′
CRKL SH2 domain:
5′-CACAGAGCTCGCCACCATGTCCTCCGCCAGGTTCGACTCCT-3′
5′-CACACTCGAGTTCCAGGTTATCTTCTGCTGTAGGC-3′
CRKL SH3 (N) domain:
5′-CACAGAGCTCGCCACCATGGGATCTGTCTCAGCACCCAACC-3′
5′-CACACTCGAGATTCTGTGTGGATGGCAAAGGGGTG-3′
CRKL SH3 (C) domain:
5′ CACAGAGCTCGCCACCATGAGATCCTCACCACACGGAAAGCATG-3′
5′ CACACTCGAGGTTTTCATCTGGGTTTTGAGGGTC-3′
CRKL-SH3-Δ1:
5′-CACCCCTTTGCCATCCACACAGAATTTGGCATTAGAGGTTGGTGACATCG-3′
5′ CGATGTCACCAACCTCTAATGCCAAATTCTGTGTGGATGGCAAAGGGGTG-3′
CRKL-SH3-Δ2:
5′ CCCTGTGCTTATGACAAGACTGCCGAAGGCGAAGTGAACGGGCGCAAA-3′
5′ TTTGCGCCCGTTCACTTCGCCTTCGGCAGTCTTGTCATAAGCACAGGG-3′
CRKL-SH3-Δ3:
5′-CAAGGATGAATATAAATGGCCAGTGGTTTGACCCTCAAAACCCAGATGAAAACG-3′
5′-CGTTTTCATCTGGGTTTTGAGGGTCAAACCACTGGCCATTTATATTCATCCTTG-3′
CRKL-SH3-Δ:
5′-CACCCCTTTGCCATCCACACAGAATTTTGACCCTCAAAACCCAGATGAAAACG-3′
5′CGTTTTCATCTGGGTTTTGAGGGTCAAAATTCTGTGTGGATGGCAAAGGGGTG-3′
PSI domain:
5′-CACAGAGCTCGCCACCATGAATTTACAACCAATTTTCTGG-3′
5′-CACACTCGAGTGTTCCTTTGCTACGGTTGGTTAC-3′
PSI linear form:
5′-CATGGGCGAATTCACAAATTCAACATTTTTACAGGAAGGAATGCCTACTTCTGCACGAC-3′
5′-TCGAGTCGTGCAGAAGTAGGCATTCCTTCCTGTAAAAATGTTGAATTTGTGAATTCGCC-3′
PSI cyclic form:
5′-CACACCATGGGCGAATTCTGTTTAAAAGCAAATGCCAAATCATG-3′
5′-CACACTCGAGGCAACCCTTCTTTTTTAAGGCTTC-3′

### Example 5

### Molecular Imaging Shows that CRKL can Localize Outside Cells

Consistent with the line of evidence presented above, molecular imaging demonstrated the co-localization of CRKL and β₁ integrin. Collectively, these studies indicate a specific molecular interaction between CRKL and β₁ integrin at the cell surface. Because the tumor homing peptide binds to the surface of DU145 prostate cancer cells, the possibility that CRKL may also localize to the outside of the cell membrane was investigated. FACS analysis of DU145 cells demonstrated cell surface labeling with an anti-CRKL antibody relative to controls (FIG. 6A). Cell surface labeling was also confirmed by immunofluorescence staining and confocal imaging studies under non-permeabilized conditions. Cell surface localization at the ultrastructural level was also studied by scanning and transmission electron microscopy (TEM; FIG. 6B) under non-permeabilized conditions as well. These classical imaging approaches again yielded cell surface labeling of CRKL (FIG. 6B). Two additional biochemical methodologies were also used to confirm cell membrane localization of CRKL: cell surface labeling by biotinylation and detergent membrane fractionation. With both independent methods, CRKL was found to be present on the cell membrane. For these studies, antibodies against unrelated intracellular proteins served as negative controls. Taken together, these results show that the CRKL protein—in addition to its well-known cytoplasmic presence—is also localized on the cell surface.

### Cell Surface and Membrane Localization

Cell surface labeling with biotin was done as described (Monferran et al., 2004). Briefly, DU145 cells were labeled with membrane impenetrable Biotin-LC-LC-NHS EZ link (Pierce). The biotinylated membrane proteins were washed, solubilized, and clarified followed by immunoprecipitation with streptavidin beads (Pharmacia). Western blots were performed with the appropriate antibodies as indicated. Phage cell surface binding assays were performed on DU145 cells as described, through the Biopanning and Rapid Analysis of Selective Interactive Ligands (BRASIL) methodology (Giordano et al., 2001). Membrane fractionation was performed as described (Mintz et al., 2003). Immunoblots were probed with the appropriate antibodies as indicated. Confocal images were acquired on an LSM 510 (Carl Zeiss) confocal microscope. DU145 cells were grown on fibronectin-coated slides, fixed with 4% paraformaldehyde (PFA) and labeled with the appropriate antibodies (polyclonal CRKL antibody and monoclonal AHSG antibody). The electron microscopy images were acquired at the High Resolution Electron Microscopy Core Facility (JSM 5900 scanning and JEM 1010 transmission electron microscopes). Gold nanoparticle antibody-conjugates were prepared by mixing 20-25 ηm or 40-45 ηm gold in sodium borate. Gold-coupled nanoparticles were verified by TEM analysis. DU145 cells were labeled on ice with appropriate antibodies (monoclonal anti-CRKL, anti-β₁ integrin, and anti-AHSG antibodies) followed by secondary conjugated-fluorescent antibodies and analyzed by FACS.

### Example 6

### Functional Studies and Potential CRKL Transport Mechanisms

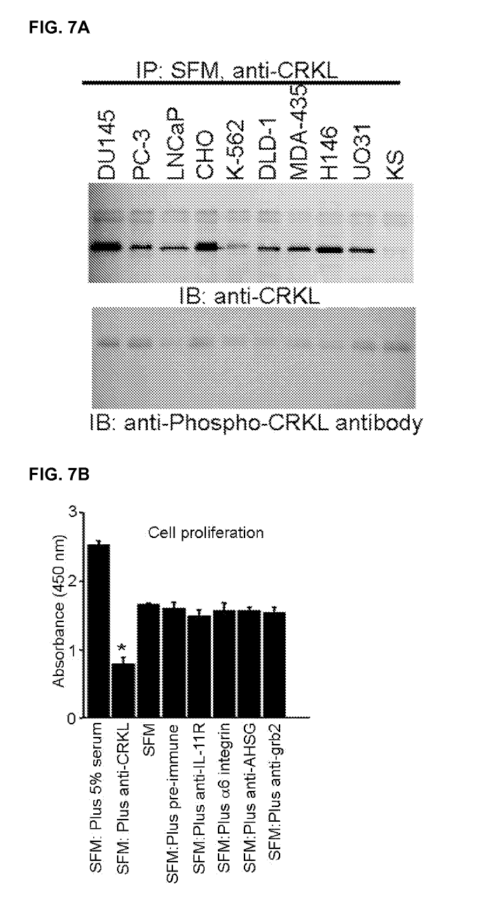

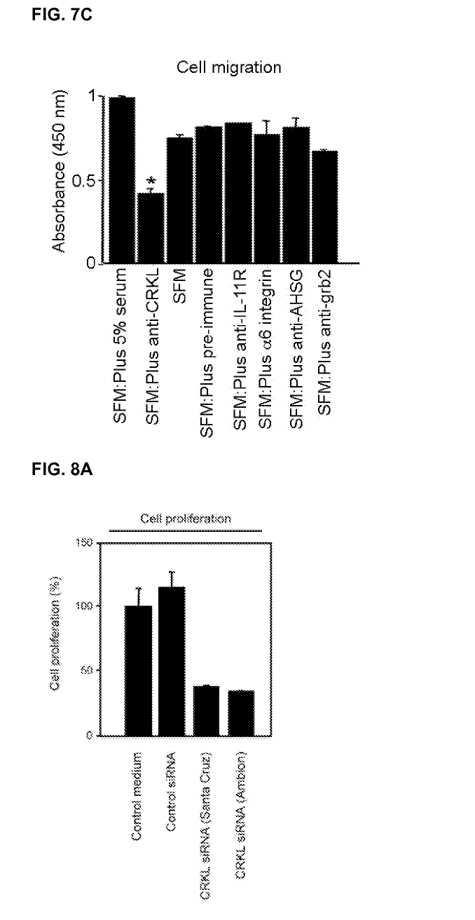

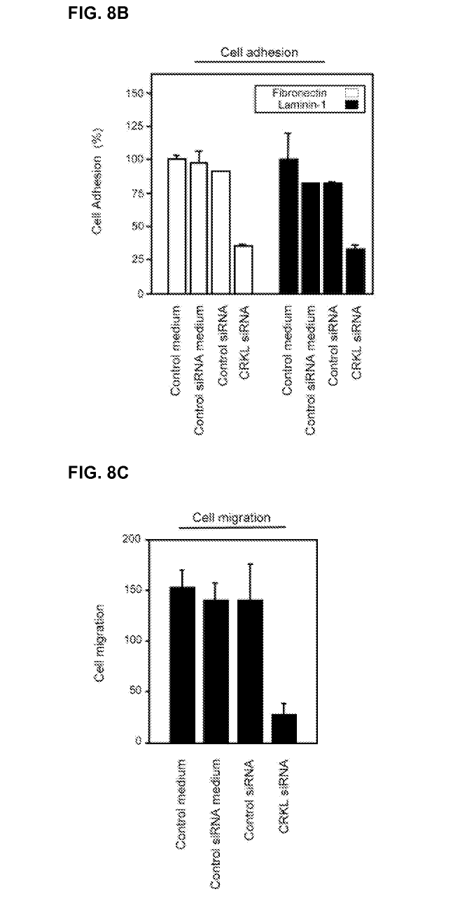

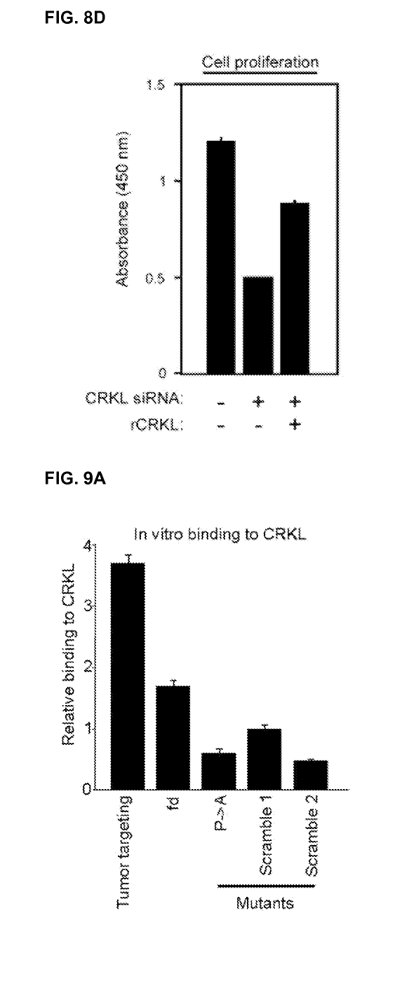

Since CRKL does not have a classic transmembrane domain, studies to evaluate whether or not CRKL is secreted from tumor cells were undertaken. Results showed that DU145 cells cultured in a serum-free medium do secrete the unphosphorylated form of CRKL. In contrast, CRKL was not detected in control cell media as shown by immunoprecipitation either with anti-CRKL or with several control antibodies. To assess the generality of these observations, a panel of tumor cell lines in serum-free media were studied and found that they also secrete unphosphorylated CRKL (FIG. 7A), thus indicating that this phenomenon is unlikely to be cell type-specific. Next, studies were undertaken to determine whether the CRKL secretion had any detectable effect(s) on cell proliferation and migration. In order to prove specificity, an anti-CRKL neutralizing antibody was added to the serum-free medium of DU145 cells in culture. Results show that the anti-CRKL antibody does neutralize extracellular CRKL and reduced cell proliferation (FIG. 7B) and cell migration (FIG. 7C); several control antibodies or pre-immune serum did not yield detectable effects on cell proliferation or migration (FIG. 7B,C). To further understand the biological role of CRKL in the context of tumor cells, CRKL knockdown by siRNA technology was employed; again, it was found that significant reductions in cell proliferation, adhesion, and migration resulted when CRKL expression is reduced (FIG. 8A-C). As an additional control, it was shown that the decrease in cell proliferation could be rescued by exogenous CRKL (FIG. 8D) and only background levels of apoptosis (less than 1%) were detected in the CRKL siRNA knockdown cells or in serum-free medium cultured cells. CRKL siRNA knockdown in cells also was found to reduced binding of the tumor-homing phage to the cells, suggesting that the tumor-homing phage may bind through secreted CRKL.

Given that CRKL does not have a hydrophobic N-terminal sequence for protein secretion via the endoplasmic reticulum and Golgi-dependent secretory pathway (Walter et al., 1984), it was determined whether classic secretory inhibitors might prevent cell release of CRKL. Results of these studies showed that inhibitors such as brefeldin A and thapsigargin do not appear to inhibit CRKL secretion; in contrast, it was found that glybenclamide, an inhibitor of adenosine triphosphate-binding cassette (ABC)-transporters, does prevent CRKL release. There are at least four known processes through which a protein—including certain growth factors and cytokines—can be secreted without a classic signal peptide (Nickel, 2003; Prudovsky et al., 2003). These data suggest that CRKL may be secreted via a non-classic export pathway by using ABC-transporters. Of note, other biologically active growth factors such as interleukin-1β and macrophage migration inhibitory factor also use ABC-transporter systems (Marty et al., 2005; Flieger et al., 2003; Hamon et al., 1997), suggesting a plausible active transport working hypothesis. However, results shown provided here do not exclude the possibility that cell death—either as part of clonal selection during malignant progression or after cytotoxic chemotherapy—could also generate extracellular CRKL within the tumor microenvironment. To gain further insight regarding this protein complex, binding assays were designed to detail the biochemical interactions among CRKL, β₁ integrins, and the PSI domain-mimic peptide. By using two different versions of recombinant CRKL (i.e., gst and his-tag), results suggest that CRKL can actually homodimerize, perhaps through the SH3 domains (Kishan et al., 1997; Kristensen et al., 2006). Because intracellular CRKL is implicated in both MAP kinase and integrin-mediated pathways (Li et al., 2003; Uemura et al., 1999), phosphorylated proteins in these two pathways were examined. Several phosphorylated proteins including paxillin, p130Cas, Erk1, Erk2, and Elk1, and even CRKL itself were found when recombinant CRKL was added to tumor cells in vitro. Moreover, when exogenous recombinant CRKL was added to β₁ integrin siRNA knockdown cells, reduced phosphorylated Erk1 and Erk2 proteins were found, indicating that ERK pathway activation by exogenous rCRKL is dependent on the expression of β₁ integrin. Positive control mitogens served to activate the MAP kinase-dependent pathway and stimulate DU145 tumor cells in order to show that the overall efficiency of the cell signaling machinery was maintained in CRKL siRNA knockdown cells. These studies demonstrated that secreted CRKL can activate integrin-mediated and MAP kinase pathways.

siRNA Studies

CRKL (mRNA accession # NM_{—}005207), β₁ integrin (mRNA accession # NM_{—}002211), and control siRNAs were purchased (Santa Cruz, Ambion and Dharmacon). siRNA oligonucleotides sequences and corresponding manufacturers are summarized in (Table 3). Oligofectamine (Invitrogen) or DharmaFect (Dharmacon) were used to transfect siRNAs into DU145 cells (1-2×10⁵ cells per well). The transfected cells were incubated for 48-72 hours prior to processing. No cell death or apoptosis was observed in the transfected cells as determined by Annexin-V staining (Roche). Transfected cells were harvested and lysed in the presence of protease inhibitors. The tumor-homing phage binding activity was also examined in the CRKL knockdown cells. For rescue experiments, up to 1.5 μg of recombinant CRKL was exogenously added to CRKL siRNA transfected cells. Exogenous His-tag recombinant CRKL protein (400 ηg/ml) or control proteins (EGF, 200 ηg/ml; MIF, 300 ηg/ml; PMA, 300 ηg/ml) were used in β₁ integrin knockdown experiments. Equal amounts of protein were loaded and resolved by SDS-PAGE followed by Western blot analysis with the appropriate antibodies. Cell proliferation assays were performed with WST-1 (Roche). Cell migration assays were performed in a Boyden chamber assay (Corning).

TABLE 3
siRNAs

Santa Cruz, CRKL:
GUCGUAUUGUCAAAGAGUATT (SEQ ID NO: 54)
GUAGCAGACAACACACAAATT (SEQ ID NO: 55)
CAGCAGACCUAGAAAUGUATT (SEQ ID NO: 56)
Dharmacon, CRKL:
CCGAAGACCUGCCCUUUAAUU (SEQ ID NO: 57)
GAAGAUAACCUGGAAUAUGUU (SEQ ID NO: 58)
GUCACAAGGAUGAAUAUAAUU (SEQ ID NO: 59)
AAUAGGAAUUCCAACAGUUUU (SEQ ID NO: 60)
Ambion, CRKL:
GGUAUCCAAGCCCACCAAUTT (SEQ ID NO: 61)
GGAUGAAUAUAAAUGGCCATT (SEQ ID NO: 62)
Santa Cruz, β₁ integrin:
GAGAUGAGGUUCAAUUUGATT (SEQ ID NO: 63)
GAUGAGGUUCAAUUUGAAATT (SEQ ID NO: 64)
GUACAGAUCCGAAGUUUCATT (SEQ ID NO: 65)
Dharmacon, control:
AUGAACGUGAAUUGCUCAAUU (SEQ ID NO: 66)

### Immunoprecipitation Assays

Cells were synchronized for 24-48 hours in RPMI serum-free medium followed by a spin and filtration through a 0.22 μm filter, and pre-absorbed to control antibodies. An equal volume of PBS was added to the recovered supernatant prior to immunoprecipitating with the appropriate antibodies. Brefeldin A, glybenclamide, and thapsigargin were used for secretion inhibition studies. Cells were incubated with the compounds in serum-free medium for 9 hours prior to immunoprecipitation with the appropriate antibodies. No detergents were used in the immunoprecipitation studies. By using the methodology described above, CRKL is found in a free soluble state and not in vesicles.

### Example 7

### CRKL-Mediated Ligand-Directed Tumor Targeting

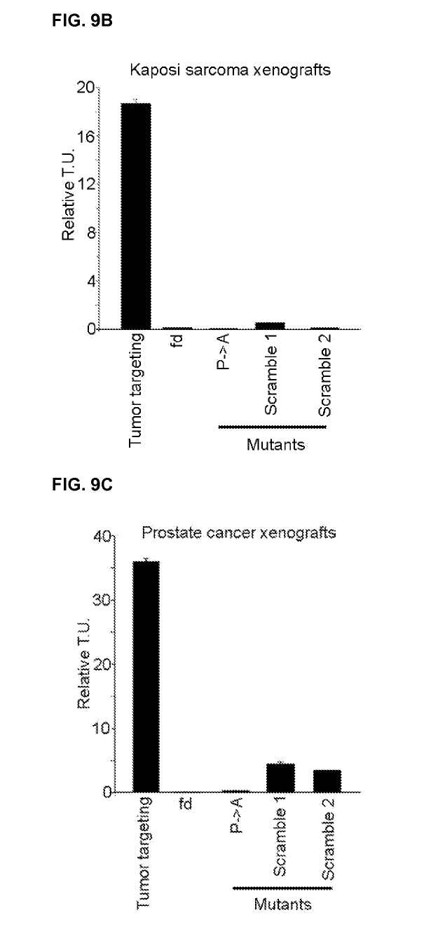

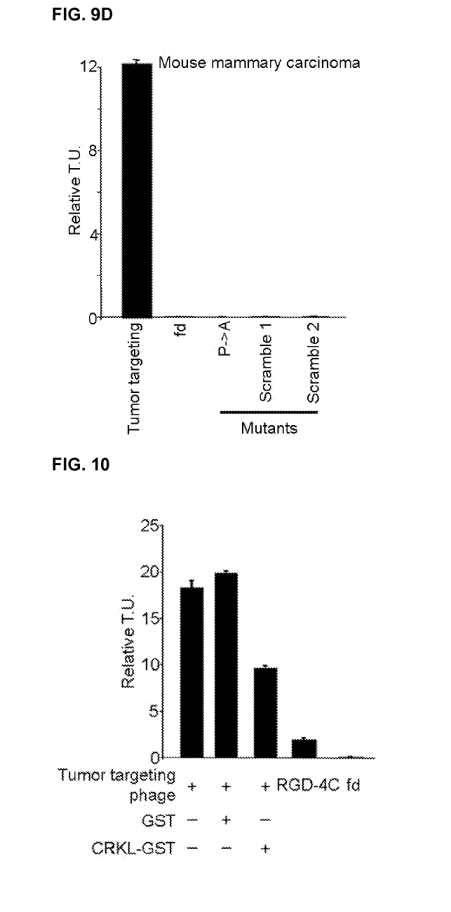

The tumor targeting attributes of CRKL-binding phage in tumor-bearing mice was evaluated next. First, phage constructs displaying either the selected CRKL-binding peptide or a panel of control (mutant or scrambled) peptides were designed and produced. Phage clones were tested in human tumor xenografts (Kaposi sarcoma KS1767 cells and prostate carcinoma DU145 cells) and an isogenic mouse tumor model (EF43-FGF4 mammary carcinoma). Marked and specific tumor homing was observed after systemic administration of the CRKL-binding phage; in contrast, control constructs showed no localization to tumors (FIG. 9A-D). As shown in FIG. 9, mutated peptide sequences P->A (YRCTLNSAFFWEDMTHECHA; SEQ ID NO:25), scramble 1 (YRFCTSPFHEWHLENTDMCA; SEQ ID NO:26) and scramble 2 (YRECTDSPHEFHLWNTMCAF; SEQ ID NO:27) demonstrated no tumor targeting Additional studies with peptides comprising a FFW->AAA mutation adjacent to the proline residue in SEQ ID NO:1 (YRCTLNSPAAAEDMTHECHA; SEQ ID NO:28) or two other scrambled sequences (#3: YRCETDSPHEFHLWNTMCAF; SEQ ID NO:29 and #4: YRCETDSPHEFHLWNTFCAM; SEQ ID NO:30) also showed no tumor targeting activity for the mutants. Furthermore, studies showed that tumor homing is inhibited when the CRKL-binding phage is pre-incubated with recombinant CRKL prior to administering to tumor-bearing mice (FIG. 10). It was also found that CRKL-binding phage was at least 10-fold higher when compared to an αv integrin-binding phage (peptide sequence CDCRGDCFC (SEQ ID NO:31), termed RGD-4C) which is typically used as a positive control for this type of experiment (Hajitou et al., 2006; Arap et al., 1998; Pasqualini et al., 2001; Giordano et al., 2001; Javadpour et al., 1996; Ellerby et al., 1999; Pasqualini et al., 1997).

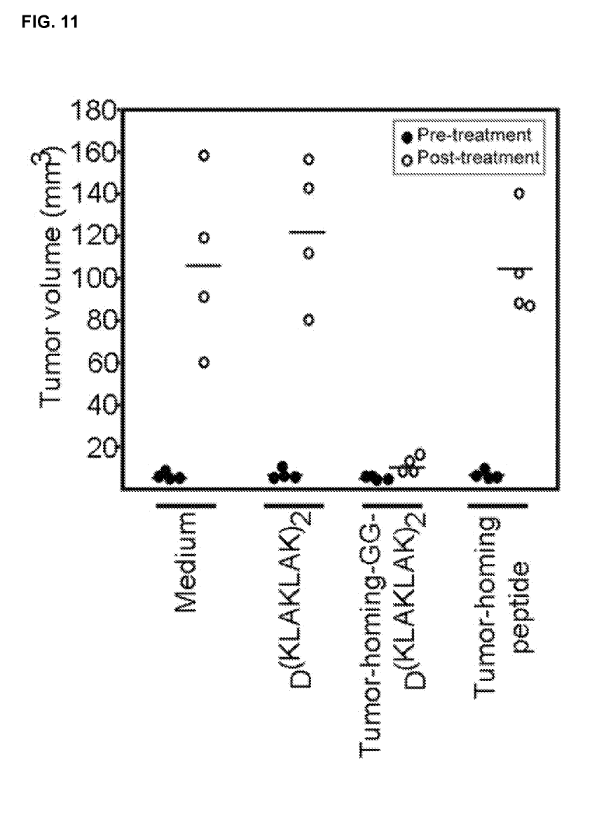

Finally, a pilot pre-clinical trial that included four cohorts of size-matched tumor-bearing mice was designed and conducted (FIG. 11). Animals received the following reagents: (i) the synthetic PSI domain-mimic peptide, (ii) the synthetic PSI domain-mimic peptide linked to a pro-apoptotic peptidomimetic (Arap et al., 2004; Ellerby et al., 1999; Kolonin et al., 2004; Zurita et al., 2004) in order to induce targeted apoptosis upon receptor-mediated internalization, (iii) the synthetic pro-apoptotic peptidomimetic alone, or (iv) vehicle alone. All peptides or peptidomimetics were administered at equimolar concentrations. These in vivo results showed that, under the experimental conditions evaluated, the PSI domain-mimic peptide had no detectable effect in tumor growth, whereas the targeted pro-apoptotic peptidomimetic significantly inhibited tumor growth.

### In Vivo Tumor Targeting and Inhibition

In vivo targeting experiments with phage were performed as described (Hajitou et al., 2006; Arap et al., 1998; Arap et al., 2004; Kolonin et al., 2004; Marchio et al., 2004). Animals used in experiments were: Male nude mice bearing human DU145 xenografts or female nude mice with human Kaposi sarcoma KS1767-derived xenografts subcutaneously and immunocompetent Balb/c female mice bearing EF43-FGF4-derived breast tumors orthotopically in the mammary fat pad. Briefly, mice bearing tumors (˜8 mm) were anesthetized and injected intravenously via tail vein with 5×10¹⁰ T.U. per mouse of wild-type YRCTLNSPFFWEDMTHECHA (SEQ ID NO:1)-phage, or negative controls: fd-tet phage (insertless) and scrambled YRFCTSPFHEWHLENTDMCA (SEQ ID NO:26)-phage, YRECTDSPHEFHLWNTMCAF (SEQ ID NO:27)-phage, YRCETDSPHEFHLWNTMCAF (SEQ ID NO:29)-phage, YRCETDSPHEFHLWNTFCAM (SEQ ID NO:30)-phage or mutants YRCTLNSAFFWEDMTHECHA (SEQ ID NO:25)-phage and YRCTLNSPAAAEDMTHECHA (SEQ ID NO:28)-phage. Cohorts of two mice with size-matched tumors received each set of phage clones. After 24 hours, tumors were dissected from each mouse and phage recovered by bacterial infection and normalized by weight of tissue. The experiments were repeated twice for each tumor model. For the inhibition of tumor targeting, the CRKL homing phage was first incubated with the recombinant gst-CRKL or control gst protein for 30 minutes at 37° C., then intravenously administered into prostate tumor-bearing mice. The fd phage served a negative control and the RGD-4C (Hajitou et al., 2006; Arap et al., 1998) served as a positive control. Only minimal background apoptosis (<1% of the total cells) were detected by TUNEL staining (Promega) on paraffin-embedded tumor tissue sections.

### Therapy in Tumor-Bearing Mice

DU145 tumor-bearing mice were size-matched and divided into individual cohorts (n=4 mice per group). The tumor-homing peptide YRCTLNSPFFWEDMTHECHA (SEQ ID NO:1) was synthesized fused with the proapoptotic motif _{D}(KLAKLAK)₂. Unconjugated peptide YRCTLNSPFFWEDMTHECHA (SEQ ID NO:1) or _{D}(KLAKLAK)₂ served as controls. The synthetic peptides were systemically administered and tumor volumes measured as described (Arap et al., 1998; Arap et al., 2004).

### MRI Imaging Using CRKL Targeting Peptides

Gold/Phage imidazole hydrogels were formed with tumor-targeting phage (targeting CRKL) and insertless phage as a control. Iron-oxide was incorporated into these hydrogels at a final volume of 30% (by volume). This hydrogel preparation was used in a subsequent MRI-study in which three prostate tumor-bearing mice (DU145) were injected intra-tumorally with equivalent amounts of AuFe only, untargeted hydrogels (with insertless phage) containing AuFe and CRKL-targeted hydrogels containing AuFe. The negative contrast mediated by the iron-core can clearly be seen and quantified using MRI.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 5,021,236
U.S. Pat. No. 5,889,155
U.S. Patent Publn. 20040023415
Arap et al., Cancer Cell, 6:275-284, 2004.
Arap et al., Cancer Res., 55(6):1351-1354, 1995.
Arap et al., Nat. Med., 8:121-127, 2002.
Arap et al., Science, 279:377-380, 1998.
Arnaout et al., Annu. Rev. Cell Dev. Biol., 21:381-410, 2005.
Baichwal and Sugden, In: Gene Transfer, Kucherlapati (Ed.), Plenum Press, NY, 117-148, 1986.
Bakhshi et al., Cell, 41(3):899-906, 1985.
Barany and Merrifield, In: The Peptides, Gross and Meienhofer (Eds.), Academic Press, NY, 1-284, 1979.
Bedi et al., Blood, 83:2038-2044, 1994.
Blume-Jensen and Hunter, Nature, 411:355-365, 2001.
Caldas et al., Cancer Res., 54:3568-3573, 1994.
Cardo-Vila et al., Mol. Cell, 11:1151-1162, 2003.
Cheng et al., Cancer Res., 54(21):5547-5551, 1994.
Cho and Stahelin, Annu. Rev. Biophys. Biomol. Struct., 34:119-151, 2005.
Cleary and Sklar, Proc. Natl. Acad. Sci. USA, 82(21):7439-7443, 1985.
Cleary et al., J. Exp. Med., 164(1):315-320, 1986.
Coffin, In: Virology, Fields et al. (Eds.), Raven Press, NY, 1437-1500, 1990.
Conner and Schmid, Nature, 422:37-44, 2003.
Culver et al., Science, 256(5063):1550-1552, 1992.
Ellerby et al., Nat. Med., 5:1032-1038, 1999.
Flieger et al., FEBS Lett., 551:78-86, 2003.
Giordano et al., Nat. Med., 7:1249-1253, 2001.
Goldstein et al., Clin. Cancer Res., 1:1311-1318, 1995.
Gomez-Foix et al., J. Biol. Chem., 267:25129-25134, 1992.
Goodman & Gilman's “The Pharmacological Basis of Therapeutics
Graham and Prevec, In: Methods in Molecular Biology: Gene Transfer and Expression Protocol, Murray (Ed.), Humana Press, Clifton, N.J., 7:109-128, 1991.
Grunhaus and Horwitz, Seminar in Virology, 3:237-252, 1992.
Hajitou et al., Cell, 125:385-398, 2006.
Hamon et al., Blood, 90:2911-2915, 1997.
Hermonat and Muzycska, Proc. Natl. Acad. Sci. USA, 81:6466-6470, 1984.
Herz and Gerard, Proc. Natl. Acad. Sci. USA, 90:2812-2816, 1993.
Hollstein et al., Science, 253(5015):49-53, 1991.
Hovanessian et al., Exp. Cell Res., 261:312-328, 2000.
Hunter, Cell, 100:113-127, 2000.
Hussussian et al., Nat. Genet., 8(1):15-21, 1994.
Javadpour et al., J. Med. Chem., 39:3107-3113, 1996.
Juliano et al., Biochem. Soc. Trans., 32:443-446, 2004.
Kamb et al., Nat. Genet., 8(1):23-26, 1994.
Kamb et al., Science, 2674:436-440, 1994.
Kang et al., EMBO J., 19:2889-2899, 2000.
Kato et al., J. Biol. Chem., 275:37481-37487, 2000.
Kerr et al., Br. J. Cancer, 26(4):239-257, 1972.
Kishan et al., Nat. Struct. Biol., 4:739-743, 1997.
Kolonin et al., Nat. Med., 10:625-632, 2004.
Kristensen et al., EMBO J. 25:785-797, 2006.
Le Gal La Salle et al., Science, 259:988-990, 1993.
Levrero et al., Gene, 101:195-202, 1991.
Li et al., Mol. Cell. Biol., 23:2883-2892, 2003.
Mandava et al., Proteomics, 4:1439-1460, 2004.
Mann et al., Cell, 33:153-159, 1983.
Manning et al., Science, 298:1912-1934, 2002.
Marchio et al., Cancer Cell, 5:151-162, 2004.
Martin et al., Science, 296:1652-1653, 2002.
Marty et al., Glia, 49:511-519, 2005.
Mayer, J. Cell Sci., 114:1253-1263, 2001.
McGahon et al., Blood, 83:1179-1187, 1994.
Merrifield, Science, 232(4748):341-347, 1986.
Mintz et al., Nat. Biotechnol., 21:57-63, 2003.
Mochly-Rosen, Science, 268:247-251, 1995.
Monferran et al., EMBO J., 23:3758-3768, 2004.
Mori et al., Cancer Res., 54(13):3396-3397, 1994.
Mould et al., J. Biol. Chem., 280:4238-4246, 2005.
Nickel, Eur. J. Biochem., 270:2109-2119, 2003.
Nicolas and Rubinstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176, 1987.
Nobori et al., Nature, 368(6473):753-756, 1994.
Okamoto et al., Proc. Natl. Acad. Sci. USA, 91(23):11045-11049, 1994.
Orlow et al., Cancer Res, 54(11):2848-2851, 1994.
Orlow et al., Int. J. Oncol., 15(1):17-24, 1994.
Paskind et al., Virology, 67:242-248, 1975.
Pasqualini and ruoslahti, Nature, 380:364-636, 1996.
Pasqualini et al., In: Phage Display: A Laboratory Manual, Barbas III et al. (Eds.), 22.1-22.24, Cold Spring Harbor Lab. Pres., NY, 2001.
Pasqualini et al., Nat. Biotechnol., 15:542-546, 1997.
Pawson and Scott, Science, 278:2075-2080, 1997.
Pendergast et al., Cell, 75:175-185, 1993.
Physicians Desk Reference
Prudovsky et al., J. Cell Sci., 116:4871-4881, 2003.
Puil et al., EMBO J., 13(4):764-773, 1994.
Ragot et al., Nature, 361:647-650, 1993.
Remington's Pharmaceutical Sciences” 15^{th} ed., pp 1035-1038 and 1570-1580
Remington's Pharmaceutical Sciences, 18th Ed. Mack Printing Company, pp. 1289-1329, 1990.
Ridgeway, In: Vectors: A Survey of Molecular Cloning Vectors and Their Uses, Rodriguez et al. (Eds.), Stoneham: Butterworth, 467-492, 1988.
Rosenfeld et al., Science, 252:431-434, 1991.
Rosenfeld, et al., Cell, 68:143-155, 1992.
Serrano et al., Nature, 366:704-707, 1993.
Serrano et al., Science, 267(5195):249-252, 1995.
Shi et al., J. Biol. Chem., 280:30586-30593, 2005.
Shin et al., J. Biol. Chem., 278:7607-7616, 2003.
Sicheri et al., Nature, 385:602-609, 1997.
Sinclair and O'Brien, J. Biol. Chem., 277:2876-2885, 2002.
Skorski et al., Blood, 86:726-736, 1995.
Skorski et al., J. Exp. Med., 179:1855-1865, 1994.
Skorski et al., Proc. Natl. Acad. Sci. USA, 91:4504-4508, 1994.
Smith and Scott, Methods Enzymol., 217:228-257, 1993.
Stewart and Young, In: Solid Phase Peptide Synthesis, 2d. ed., Pierce Chemical Co., 1984.
Szczulik et al., Science, 253:562-265, 1991.
Takagi et al., Cell, 110:599-611, 2002.
Tam et al., J. Am. Chem. Soc., 105:6442, 1983.
Tari et al., Blood, 84:601-607, 1994.
Temin, In: Gene Transfer, Kucherlapati (Ed.), NY, Plenum Press, 149-188, 1986.
ten Hoeve et al., Blood, 84:1731-1736, 1994.
ten Hoeve et al., Cancer Res., 54:2563-2567, 1994.
ten Hoeve et al., Oncogene, 8:2469-2474, 1993.
Tsujimoto and Croce, Proc. Natl. Acad. Sci. USA, 83(14):5214-5218, 1986.
Tsujimoto et al., Nature, 315:340-343, 1985.
Tsujimoto et al., Science, 228(4706):1440-1443, 1985.
Uemura et al., Oncogene, 18:3343-3353, 1999.
Walter et al., Cell, 38:5-8, 1984.
Weinberg, Science, 254(5035):1138-1146, 1991.
Wong et al., Gene, 10:87-94, 1980.
Xu et al., Nature, 385:595-602, 1997.
Zurita et al., Cancer Res., 64:435-439, 2004.

1. An isolated tumor targeting peptide comprising a CRKL binding motif, said motif defined as:
a) being from 6 to 20 amino acids in length;
b) having a degree of similarity to a corresponding best fit sequence alignment to β1 integrin (SEQ ID NO:47) of at least 25%; and wherein the targeting peptide is 100 amino acids or less in length and binds under physiological conditions to cells expressing CRKL.

2. The peptide of claim 1, wherein the CRKL binding motif has a degree of similarity to a best fit sequence alignment to β1 integrin (SEQ ID NO:47) of at least 40%.

3. The peptide of claim 1, wherein the CRKL binding motif has a degree of similarity to a best fit sequence alignment to β1 integrin (SEQ ID NO:47) of at least 50%.

4. The peptide of claim 1, wherein the CRKL binding motif has a degree of similarity to a best fit sequence alignment to β1 integrin (SEQ ID NO:47) of at least 60%.

5. The peptide of claim 1, wherein the peptide has a sequence that is not identical to a best fit sequence alignment to β1 integrin (SEQ ID NO:47).

6. The peptide of claim 1, wherein the CRKL binding motif has a best fit sequence alignment to a β1 integrin (SEQ ID NO:47) PSI domain region.

7. The peptide of claim 1, wherein the CRKL binding motif has a best fit sequence alignment to a β1 integrin (SEQ ID NO:47) PSI domain region selected from the group consisting of amino acids 10 to 29; 15 to 34; 18 to 37; 36 to 55; 39 to 58; 45 to 64; 94 to 113; 196 to 215; 198 to 213; 203 to 222; 244 to 263; 330 to 349; 377 to 396; 379 to 398; 380 to 399; 398 to 417; 400 to 419; 413 to 432; 447 to 466; 460 to 479; 460 to 479; 464 to 483; 469 to 488; 474 to 493; 475 to 494; 512 to 533; 519 to 538; 551 to 570; 574 to 593; 577 to 596; 579 to 598; 590 to 609; 596 to 615; 613 to 632; 615 to 634; 616 to 635; 644 to 663; 648 to 667; 663 to 682; 674 to 693; 682 to 701; 721 to 740; 727 to 746; and 779 to 798.

8. The peptide of claim 1, wherein the CRKL binding motif has a sequence selected from the group consisting of SEQ ID NO:1 through SEQ ID NO:46.

9. The isolated peptide of claim 1, further defined as a cyclic peptide.

10. The isolated peptide of claim 1, wherein said peptide is attached to a molecule.

11. The isolated peptide of claim 10, wherein the molecule is a protein and the peptide is conjugated or fused to the protein to form a protein conjugate, wherein the protein conjugate is not a naturally occurring protein.

12. The isolated peptide of claim 11, wherein the peptide is positioned at a terminus of the protein.

13. The isolated peptide of claim 10, wherein said molecule is a pro-apoptosis agent, an anti-angiogenic agent, a cytokine, a cytotoxic agent, a drug, a chemotherapeutic agent, a hormone, a growth factor, an antibiotic, an antibody or fragment or single chain thereof, a survival factor, an anti-apoptotic agent, a hormone antagonist, an antigen, a peptide, a protein, a diagnostic agent, a radioisotope, or an imaging agent.

14. The isolated peptide of claim 13, wherein said molecule is a pro-apoptosis agent selected from the group consisting of gramicidin; magainin; mellitin; defensin; cecropin; (KLAKLAK)₂ (SEQ ID NO:48); (KLAKKLA)₂ (SEQ ID NO:49); (KAAKKAA)₂ (SEQ ID NO:50); (KLGKKLG)₃ (SEQ ID NO:51); Bcl-2; Bad; Bak; Bax; and Bik.

15. The isolated peptide of claim 14, wherein said pro-apoptosis agent is (KLAKLAK)₂ (SEQ ID NO:48).

16. The isolated peptide of claim 15, wherein said SEQ ID NO:48 consists of D amino acids.

17. The isolated peptide of claim 13, wherein said molecule is an anti-angiogenic agent selected from the group consisting of thrombospondin, an angiostatin, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4, minocycline, endostatin XVIII, endostatin XV, the C-terminal hemopexin domain of matrix metalloproteinase-2, the kringle 5 domain of human plasminogen, a fusion protein of endostatin and angiostatin, a fusion protein of endostatin and the kringle 5 domain of human plasminogen, the monokine-induced by interferon-gamma (Mig), a fusion protein of Mig and IP10, soluble FLT-1 (fins-like tyrosine kinase 1 receptor), or kinase insert domain receptor (KDR).

18. The isolated peptide of claim 13, wherein said molecule is a cytokine selected from the group consisting of interleukin 1 (IL-1), IL-2, IL-5, IL-10, IL-11, IL-12, IL-18, interferon-γ (IF-γ), IF-α, IF-β, a tumor necrosis factor, or GM-CSF (granulocyte macrophage colony stimulating factor).

19. The isolated peptide of claim 10, wherein said peptide is attached to a macromolecular complex.

20. The isolated peptide of claim 19, wherein said complex is a virus, a bacteriophage, a bacterium, a liposome, a microparticle, a magnetic bead, a yeast cell, or a mammalian cell.

21. The isolated peptide of claim 13, wherein said peptide is attached to a virus.

22. The isolated peptide of claim 14, wherein said virus is a lentivirus, papovavirus, adenovirus, retrovirus, AAV, vaccinia virus or herpes virus.

23. The isolated peptide of claim 19, wherein said peptide is attached to a solid support.

24. The isolated peptide of claim 23, wherein the solid support is a microtiter dish or microchip.

25. A method of preparing a construct comprising obtaining a peptide in accordance with claim 1 and attaching the peptide to a molecule to prepare the construct.

26. A method of targeting the delivery of a peptide, molecule or protein to cells that express CRKL, the method comprising the steps of:
(a) obtaining a peptide according to claim 1; and
(b) administering the peptide to a cell population, wherein the population includes cells that express CRKL, to thereby deliver the molecule or protein to said cells.

27. The method of claim 26, wherein the cells that express CRKL are in a subject, the peptide or protein fusion construct is formulated in a pharmaceutically acceptable composition and the composition is administered to the subject.

28. The method of claim 27, wherein the subject is a human subject.

29. The method of claim 26, wherein the method is further defined as a detection method and the method further comprises detecting the peptide, molecule or protein that has been delivered to the cells.

30. The method of claim 26, wherein the subject has a disease or disorder and the method is further defined as a therapeutic method.

31. The method of claim 30, wherein the subject has a cancer.

32. The method of claim 31, wherein the cancer is selected from the group of cancers of the prostate, breast, sarcoma, gum, tongue, lung, skin, liver, kidney, eye, brain, leukemia, mesothelioma, neuroblastoma, head, neck, pancreatic, renal, bone, testicular, ovarian, mesothelioma, cervical, gastrointestinal, lymphoma, brain, colon and bladder.

33. The method of claim 32, wherein the cancer is prostate cancer.

34. The method of claim 32, wherein the cancer is breast cancer.

35. The method of claim 32, wherein the cancer is sarcoma.

36. The method of claim 1, wherein said motif is further defined as being from 6 to 10 amino acids in length.

37. The method of claim 1, wherein said motif is further defined as being from 14 to 20 amino acids in length.


SEQUENCE LISTING (69 sequences)

SEQ ID NO: 1 - protein, 20 aa, Homo sapiens
YRCTLNSPFFWEDMTHECHA

SEQ ID NO: 2 - protein, 20 aa, Homo sapiens
LGCMASMLREFEGATHACTQ

SEQ ID NO: 3 - protein, 20 aa, Homo sapiens
RGCTEAAGLVIGITTHQCGN

SEQ ID NO: 4 - protein, 20 aa, Homo sapiens
IGCNHPSPLGSTVVPTYCFK

SEQ ID NO: 5 - protein, 20 aa, Homo sapiens
GTCPRQFFHMQEFWPSDCSR

SEQ ID NO: 6 - protein, 20 aa, Homo sapiens
DRCVLVRPEFGRGDARLCHS

SEQ ID NO: 7 - protein, 20 aa, Homo sapiens
EGCSDIMNTAAERVTGDCSY

SEQ ID NO: 8 - protein, 20 aa, Homo sapiens
VFCCGSYCGGVEMLASRCGH

SEQ ID NO: 9 - protein, 20 aa, Homo sapiens
RECGRTVHRYPWGSPESCER

SEQ ID NO: 10 - protein, 20 aa, Homo sapiens
DACSRFLGERVDATAAGCSR

SEQ ID NO: 11 - protein, 20 aa, Homo sapiens
GNCMGLQVSELFMGPYKCRQ

SEQ ID NO: 12 - protein, 51 aa, Homo sapiens
RCLKANAKSCGECIQAGPNCGWCTNSTFLQEGMPTSARCDDLEALKKKGCP

SEQ ID NO: 13 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 14 - protein, 21 aa, Homo sapiens
CGWCTNSTFLQEGMPTSARCD

SEQ ID NO: 15 - protein, 21 aa, Homo sapiens
CTWCQKLNFTGPGDPDSIRCD

SEQ ID NO: 16 - protein, 17 aa, Homo sapiens
CAWCSDEALPLGSPRCD

SEQ ID NO: 17 - protein, 15 aa, Homo sapiens
CAYCTDEMFRDRRCN

SEQ ID NO: 18 - protein, 20 aa, Homo sapiens
CAWCSKEDFGSPRSITSRCD

SEQ ID NO: 19 - protein, 20 aa, Homo sapiens
CAWCAQENFTHPSGVGERCD

SEQ ID NO: 20 - protein, 21 aa, Homo sapiens
CAWCKQLNFTASGEAEARRCA

SEQ ID NO: 21 - protein, 20 aa, Homo sapiens
CGWCVQEDFISGGSRSERCD

SEQ ID NO: 22 - protein, 303 aa, Homo sapiens
MSSARFDSSDRSAWYMGPVSRQEAQTRLQGQRHGMFLVRDSSTCPGDYVLSVSENSRVSHYIINSLPNRRFKIGDQEFDHLPALLEFYKIHYLDTTTLIEPAPRYPSPPMGSVSAPNLPTAEDNLEYVRTLYDFPGNDAEDLPFKKGEILVIIEKPEEQWWSARNKDGRVGMIPVPYVEKLVRSSPHGKHGNRNSNSYGIPEPAHAYAQPQTTTPLPAVSGSPGAAITPLPSTQNGPVFAKAIQKRVPCAYDKTALALEVGDIVKVTRMNINGQWEGEVNGRKGLFPFTHVKIFDPQNPDENE

SEQ ID NO: 23 - protein, 13 aa, Homo sapiens
NSTFLQEGMPTSA

SEQ ID NO: 24 - protein, 58 aa, Homo sapiens
GPVFAKAIQKRVPCAYDKTALALEVGDIVKVTRMNINGQWEGEVNGRKGLFPFTHVKI

SEQ ID NO: 25 - protein, 20 aa, Homo sapiens
YRCTLNSAFFWEDMTHECHA

SEQ ID NO: 26 - protein, 20 aa, Homo sapiens
YRFCTSPFHEWHLENTDMCA

SEQ ID NO: 27 - protein, 20 aa, Homo sapiens
YRECTDSPHEFHLWNTMCAF

SEQ ID NO: 28 - protein, 20 aa, Homo sapiens
YRCTLNSPAAAEDMTHECHA

SEQ ID NO: 29 - protein, 20 aa, Homo sapiens
YRCETDSPHEFHLWNTMCAF

SEQ ID NO: 30 - protein, 20 aa, Homo sapiens
YRCETDSPHEFHLWNTFCAM

SEQ ID NO: 31 - protein, 9 aa, Homo sapiens
CDCRGDCFC

SEQ ID NO: 32 - DNA, 35 bp, Homo sapiens
cacagagctcaacaccatgtcctccgccaggttcg

SEQ ID NO: 33 - DNA, 35 bp, Homo sapiens
cacactcgagctcgttttcatctgggttttgaggg

SEQ ID NO: 34 - DNA, 41 bp, Homo sapiens
cacagagctcgccaccatgtcctccgccaggttcgactcct

SEQ ID NO: 35 - DNA, 35 bp, Homo sapiens
cacactcgagttccaggttatcttctgctgtaggc

SEQ ID NO: 36 - DNA, 41 bp, Homo sapiens
cacagagctcgccaccatgggatctgtctcagcacccaacc

SEQ ID NO: 37 - DNA, 35 bp, Homo sapiens
cacactcgagattctgtgtggatggcaaaggggtg

SEQ ID NO: 38 - DNA, 44 bp, Homo sapiens
cacagagctcgccaccatgagatcctcaccacacggaaagcatg

SEQ ID NO: 39 - DNA, 34 bp, Homo sapiens
cacactcgaggttttcatctgggttttgagggtc

SEQ ID NO: 40 - DNA, 50 bp, Homo sapiens
cacccctttgccatccacacagaatttggcattagaggttggtgacatcg

SEQ ID NO: 41 - DNA, 50 bp, Homo sapiens
cgatgtcaccaacctctaatgccaaattctgtgtggatggcaaaggggtg

SEQ ID NO: 42 - DNA, 48 bp, Homo sapiens
ccctgtgcttatgacaagactgccgaaggcgaagtgaacgggcgcaaa

SEQ ID NO: 43 - DNA, 48 bp, Homo sapiens
tttgcgcccgttcacttcgccttcggcagtcttgtcataagcacaggg

SEQ ID NO: 44 - DNA, 54 bp, Homo sapiens
caaggatgaatataaatggccagtggtttgaccctcaaaacccagatgaaaacg

SEQ ID NO: 45 - DNA, 54 bp, Homo sapiens
cgttttcatctgggttttgagggtcaaaccactggccatttatattcatccttg

SEQ ID NO: 46 - DNA, 53 bp, Homo sapiens
cacccctttgccatccacacagaattttgaccctcaaaacccagatgaaaacg

SEQ ID NO: 47 - DNA, 53 bp, Homo sapiens
cgttttcatctgggttttgagggtcaaaattctgtgtggatggcaaaggggtg

SEQ ID NO: 48 - DNA, 40 bp, Homo sapiens
cacagagctcgccaccatgaatttacaaccaattttctgg

SEQ ID NO: 49 - DNA, 34 bp, Homo sapiens
cacactcgagtgttcctttgctacggttggttac

SEQ ID NO: 50 - DNA, 59 bp, Homo sapiens
catgggcgaattcacaaattcaacatttttacaggaaggaatgcctacttctgcacgac

SEQ ID NO: 51 - DNA, 59 bp, Homo sapiens
tcgagtcgtgcagaagtaggcattccttcctgtaaaaatgttgaatttgtgaattcgcc

SEQ ID NO: 52 - DNA, 44 bp, Homo sapiens
cacaccatgggcgaattctgtttaaaagcaaatgccaaatcatg

SEQ ID NO: 53 - DNA, 34 bp, Homo sapiens
cacactcgaggcaacccttcttttttaaggcttc

SEQ ID NO: 54 - DNA, 21 bp, Homo sapiens
gucguauugucaaagaguatt

SEQ ID NO: 55 - DNA, 21 bp, Homo sapiens
guagcagacaacacacaaatt

SEQ ID NO: 56 - DNA, 21 bp, Homo sapiens
cagcagaccuagaaauguatt

SEQ ID NO: 57 - DNA, 21 bp, Homo sapiens
ccgaagaccugcccuuuaauu

SEQ ID NO: 58 - RNA, 21 nt, Homo sapiens
gaagauaaccuggaauauguu

SEQ ID NO: 59 - DNA, 21 bp, Homo sapiens
gucacaaggaugaauauaauu

SEQ ID NO: 60 - DNA, 21 bp, Homo sapiens
aauaggaauuccaacaguuuu

SEQ ID NO: 61 - DNA, 21 bp, Homo sapiens
gguauccaagcccaccaautt

SEQ ID NO: 62 - DNA, 21 bp, Homo sapiens
ggaugaauauaaauggccatt

SEQ ID NO: 63 - DNA, 21 bp, Homo sapiens
gagaugagguucaauuugatt

SEQ ID NO: 64 - DNA, 21 bp, Homo sapiens
gaugagguucaauuugaaatt

SEQ ID NO: 65 - DNA, 21 bp, Homo sapiens
guacagauccgaaguuucatt

SEQ ID NO: 66 - DNA, 21 bp, Homo sapiens
augaacgugaauugcucaauu

SEQ ID NO: 67 - protein, 22 aa, Homo sapiens
YRCTLNSPFFWEDMTHECHAGG

SEQ ID NO: 68 - DNA, 15 bp, Homo sapiens
gtgagccggctgccc

SEQ ID NO: 69 - DNA, 15 bp, Homo sapiens
ttcggccccagcggc